(19) United States
(12) Patent Application Publication
(10) Pub. No.: US 2008/0207596 A1
(43) Pub. Date: Aug. 28, 2008

(54) Novel compounds 329

(76) Inventors: Barrie Martin, Loughborough (GB); Paul Willis, Loughborough (GB)

Correspondence Address: MORGAN LEWIS & BOCKIUS LLP, 1111 PENNSYLVANIA AVENUE NW, WASHINGTON, DC, 20004, US

(21) Appl. No.: 11/812,474

(22) Filed: Jun. 19, 2007

Related U.S. Application Data
(60) Provisional application No. 60/814,935, filed on Jun. 19, 2006.

(51) Int. Cl. A61K 31/551; C07D 401/14; A61P 19/02; A61P 11/06; A61K 31/4725

(52) U.S. Cl. 514/217.07; 546/141; 514/309; 540/597

(57) ABSTRACT

The invention concerns a compound of formula (I)

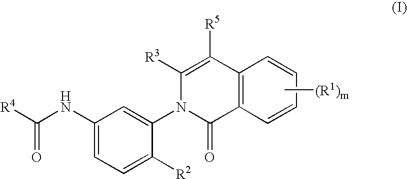

or pharmaceutically-acceptable salts thereof wherein R¹, R², R³, R⁴, R⁵ and m are as defined in the specification, processes for their preparation, pharmaceutical compositions containing them and their use in the treatment of diseases or medical conditions mediated by cytokines.

This invention relates to amide derivatives, or pharmaceutically-acceptable salts thereof which are useful as inhibitors of cytokine mediated disease. The invention also relates to processes for the manufacture of said amide derivatives, to pharmaceutical compositions containing said amide derivatives and to their use in therapeutic methods, for example by virtue of inhibition of cytokine mediated disease.

The amide derivatives disclosed in the present invention are inhibitors of the production of cytokines such as Tumour Necrosis Factor (hereinafter TNF), for example TNFα, and various members of the interleukin (hereinafter IL) family, for example IL-1, IL-6 and IL-8. Accordingly the amide derivatives of the invention will be useful in the treatment of diseases or medical conditions in which excessive production of cytokines occurs, for example excessive production of TNFα or IL-1. It is known that cytokines are produced by a wide variety of cells such as monocytes and macrophages and that they give rise to a variety of physiological effects which are believed to be important in disease or medical conditions such as inflammation and immunoregulation. For example, TNFα and IL-1 have been implicated in the cell signalling cascade which is believed to contribute to the pathology of disease states such as inflammatory and allergic diseases and cytokine-induced toxicity. It is also known that, in certain cellular systems, TNFα production precedes and mediates the production of other cytokines such as IL-1.

Abnormal levels of cytokines have also been implicated in, for example, the production of physiologically-active eicosanoids such as the prostaglandins and leukotrienes, the stimulation of the release of proteolytic enzymes such as collagenase, the activation of the immune system, for example by stimulation of T-helper cells, the activation of osteoclast activity leading to the resorption of calcium, the stimulation of the release of proteoglycans from, for example, cartilage, the stimulation of cell proliferation and to angiogenesis.

Cytokines are also believed to be implicated in the production and development of disease states such as inflammatory and allergic diseases, for example inflammation of the joints (especially rheumatoid arthritis, osteoarthritis and gout), inflammation of the gastrointestinal tract (especially inflammatory bowel disease, ulcerative colitis, Crohn's disease and gastritis), skin disease (especially psoriasis, eczema and dermatitis) and respiratory disease (especially asthma, bronchitis, allergic rhinitis, chronic obstructive pulmonary disease and adult respiratory distress syndrome), and in the production and development of various cardiovascular and cerebrovascular disorders such as congestive heart failure, acute heart failure, myocardial infarction, the formation of atherosclerotic plaques, hypertension, platelet aggregation, angina, stroke, reperfusion injury, vascular injury including restenosis and peripheral vascular disease, and, for example, various disorders of bone metabolism such as osteoporosis (including senile and postmenopausal osteoporosis), Paget's disease, bone metastases, hypercalcaemia, hyperparathyroidism, osteosclerosis, osteoperosis and periodontitis, and the abnormal changes in bone metabolism which may accompany rheumatoid arthritis and osteoarthritis. Excessive cytokine production has also been implicated in mediating certain complications of bacterial, fungal and/or viral infections such as endotoxic shock, septic shock and toxic shock syndrome and in mediating certain complications of CNS surgery or injury such as neurotrauma and ischaemic stroke. Excessive cytokine production has also been implicated in mediating or exacerbating the development of diseases involving cartilage or muscle resorption, pulmonary fibrosis, cirrhosis, renal fibrosis, the cachexia found in certain chronic diseases such as malignant disease and acquired immune deficiency syndrome (AIDS), chronic obstructive pulmonary disease, tumour invasiveness and tumour metastasis and multiple sclerosis. Excessive cytokine production has also been implicated in pain.

Evidence of the central role played by TNFα in the cell signalling cascade which gives rise to rheumatoid arthritis is provided by the efficacy in clinical studies of antibodies of TNFα (The Lancet, 1994, 344, 1125 and British Journal of Rheumatology, 1995, 34, 334).

Thus cytokines such as TNFα and IL-1 are believed to be important mediators of a considerable range of diseases and medical conditions. Accordingly it is expected that inhibition of the production of and/or effects of these cytokines will be of benefit in the prophylaxis, control or treatment of such diseases and medical conditions.

Without wishing to imply that the amide derivatives disclosed in the present invention possesses pharmacological activity only by virtue of an effect on a single biological process, it is believed that the amide derivatives inhibit the effects of cytokines by virtue of inhibition of the enzyme p38 kinase. p38 kinase, otherwise known as cytokine suppressive binding protein (hereinafter CSBP) and reactivating kinase (hereinafter RK), is a member of the mitogen-activated protein (hereinafter MAP) kinase family of enzymes which is known to be activated by physiological stress such as that induced by ionising radiation, cytotoxic agents, and toxins, for example endotoxins such as bacterial lipopolysaccharide, and by a variety of agents such as the cytokines, for example TNFα and IL-1. It is known that p38 kinase phosphorylates certain intracellular proteins which are involved in the cascade of enzymatic steps which leads to the biosynthesis and excretion of cytokines such as TNFα and IL-1. Known inhibitors of p38 kinase have been reviewed by G. J. Hanson in Expert Opinions on Therapeutic Patents, 1997, 7, 729-733. p38 kinase is known to exist in isoforms identified as p38α and p380.

It is known from International Patent Application WO 00/55153, that certain quinazolinone-benzamide derivatives are inhibitors of the production of cytokines such as TNF, and various interleukins. However, there is a need to find further compounds that possess potent cytokine inhibitory activity and have desirable pharmacological activity profiles.

The present invention provides compounds that are inhibitors of the production of cytokines such as TNF (in particular of TNFα, and various interleukins, in particular IL-1). In addition to high potency, the compounds may display advantageous pharmaceutical properties such as rapid drug absorption, rapid onset of action and reduced side effects.

According to the present invention there is provided a compound of formula (I)

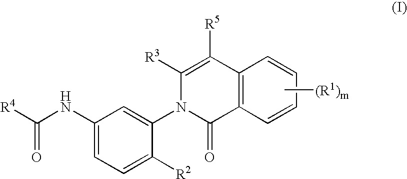

wherein
m is 0, 1 or 2;
R¹ is halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphonyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, aryl, aryl-(1-6C)alkyl, aryloxy, arylthio, arylsulphinyl, arylsulphonyl, aryl-(1-6C)alkoxy, arylamino, and wherein any aryl, heteroaryl or heterocyclyl group in a R¹ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl, and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R¹ substituent may optionally bear 1 or 2 oxo or thioxo substituents;
R² is halogeno or (1-6C)alkyl;
R³ is hydrogen, halogeno, trifluoromethyl, cyano, (1-6C)alkyl, (1-6C)alkoxy, (1-6C)alkylthio, (1-6C)alkylamino, di[(1-6C)alkyl]amino, di-[(1-6C)alkyl]amino-(2-6C)alkoxy, (1-6C)alkylamino-(2-6C)alkoxy, amino-(2-6C)alkoxy, (1-6C)alkoxy-(2-6C)alkoxy, amino-(1-6C)alkyl, di[(1-6C)alkyl]amino-(1-6C)alkyl or (1-6C)alkylamino-(1-6C)alkyl;
R⁵ is hydrogen, halogeno, trifluoromethyl, cyano, (1-6C)alkyl, (1-6C)alkoxy, (1-6C)alkylthio, (1-6C)alkylamino, di[(1-6C)alkyl]amino, di-[(1-6C)alkyl]amino-(2-6C)alkoxy, (1-6C)alkylamino-(2-6C)alkoxy, amino-(2-6C)alkoxy, (1-6C)alkoxy-(2-6C)alkoxy, amino-(1-6C)alkyl, di[(1-6C)alkyl]amino-(1-6C)alkyl or (1-6C)alkylamino-(1-6C)alkyl;
R⁴ is aryl or heteroaryl, which aryl or heteroaryl is optionally substituted by one or more substituents independently selected from halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, hydroxy(1-6C)alkylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphinoyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, heterocyclylthio, aryl, aryl-(1-6C)alkyl, aryloxy, arylthio, arylthio, arylsulphinyl, aryl-(1-6C)alkoxy, arylamino, (3-6C)cycloalkyl, (3-6C)cycloalkyl-(1-6C)alkyl, (3-6C)cycloalkyloxy, (3-6C)cycloalkylthio, (3-6C)cycloalkyl-(1-6C)alkoxy and (3-6C)cycloalkylamino, and wherein any heteroaryl, heterocyclyl, aryl or (3-6C)cycloalkyl group in a R⁴ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, (1-6C)alkylthio, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, (2-6C)alkanoyloxy, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl, and wherein any of the R⁴ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R⁴ substituent may optionally bear 1 or 2 oxo or thioxo substituents;
or a pharmaceutically-acceptable salt thereof.

It will be understood that certain compounds of the present invention may exist in solvated, for example hydrated, as well as unsolvated forms. It is to be understood that the present invention encompasses all such solvated forms.

It is to be understood that, insofar as certain of the compounds of formula (I) defined above may exist in optically active or racemic forms by virtue of one or more asymmetric carbon atoms, the invention includes in its definition any such optically active or racemic form which possesses the property of inhibiting cytokines, in particular TNF. The synthesis of optically active forms may be carried out by standard techniques of organic chemistry well known in the art, for example by synthesis from optically active starting materials or by resolution of a racemic form. Similarly, inhibitory properties against TNF may be evaluated using the standard laboratory techniques referred to hereinafter.

In the present specification, the term (1-6C)alkyl includes straight-chain and branched-chain alkyl groups such as propyl, isopropyl and tert-butyl. References to individual alkyl groups such as “propyl” are specific for the straight-chain version only, references to individual branched-chain alkyl groups such as “isopropyl” are specific for the branched-chain version only.

In the present specification, the term (3-6C)cycloalkyl denotes non-aromatic carbon ring structures, for example cyclopropyl, cyclobutyl, cyclopentyl, cyclopentenyl and cyclohexyl. References to individual cycloalkyl groups such as “cyclopentyl” are specific for that 5-membered ring only.

In the present specification the term ‘aryl’ denotes aromatic carbon ring structures, for example phenyl, indenyl, indanyl, naphthyl, tetrahydronaphthyl or fluorenyl.

In the present specification the term ‘heteroaryl’ denotes aromatic ring structures, for example an aromatic 5- or 6-membered monocyclic ring, a 9- or 10-membered bicyclic ring or a 13- or 14-membered tricyclic ring each comprising at least one ring heteroatom selected from oxygen, nitrogen and sulphur, or an N-oxide, S-oxide or S-dioxide thereof, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl, naphthyridinyl, carbazolyl, dibenzofuranyl, dibenzothiophenyl, S,S-dioxodibenzothiophenyl, xanthenyl, dibenzo-1,4-dioxinyl, phenoxathiinyl, phenoxazinyl, dibenzothiinyl, phenothiazinyl, thianthrenyl, benzofuropyridyl, pyridoindolyl, acridinyl or phenanthridinyl.

In the present specification the term ‘heterocyclyl’ denotes non-aromatic ring structures, for example a 3- to 10-membered monocyclic or bicyclic ring or a 5- to 7-membered monocyclic ring each comprising at least one ring heteroatom selected from oxygen, nitrogen and sulphur, or an N-oxide, S-oxide or S-dioxide thereof; for example oxiranyl, oxetanyl, azetidinyl, tetrahydrofuranyl, tetrahydropyranyl, pyrrolinyl, pyrrolidinyl, imidazolinyl, imidazolidinyl, pyrazolinyl, pyrazolidinyl, 1,1-dioxidoisothiazolidinyl, morpholinyl, thiomorpholinyl, tetrahydro-1,4-thiazinyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl or benzo derivatives thereof such as 2,3-dihydrobenzofuranyl, 2,3-dihydrobenzothienyl, indolinyl, isoindolinyl, chromanyl and isochromanyl; examples of heterocyclyl rings bearing 1 or 2 oxo or thioxo substituents are, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

Examples of suitable values for substituent groups in the present specification are as follows:
for halogeno: fluoro, chloro, bromo and iodo;
for (1-6C)alkyl: methyl, ethyl, propyl, isopropyl and tert-butyl;
for (2-6C)alkenyl: vinyl and allyl;
for (2-6C)alkynyl: ethynyl and 2-propynyl;
for (2-6C)alkanoyl: acetyl and propionyl;
for (1-6C)alkoxy: methoxy, ethoxy, propoxy, isopropoxy and butoxy;
for (1-6C)alkylthio: methylthio, ethylthio and propylthio;
for (1-6C)alkylsulphinyl: methylsulphinyl, ethylsulphinyl and propylsulphinyl;
for (1-6C)alkylsulphonyl: methylsulphonyl, ethylsulphonyl and propylsulphonyl;
for hydroxy-(2-6C)alkoxy: 2-hydroxyethoxy, 3-hydroxypropoxy, 2-hydroxy-1-methylethoxy, 2-hydroxy-2-propoxy and 4-hydroxybutoxy;
for amino-(2-6C)alkoxy: 2-aminoethoxy, 2-amino-1-methylethoxy, 3-aminopropoxy, 2-amino-2-methylpropoxy and 4-aminobutoxy;
for cyano-(2-6C)alkoxy: 2-cyanoethoxy and 3-cyanopropoxy;
for (1-6C)alkylamino: methylamino, ethylamino and propylamino;
for di-[(1-6C)alkyl]amino: dimethylamino, diethylamino and N-ethyl-N-methylamino;
for (1-6C)alkylamino-(2-6C)alkoxy: 2-methylaminoethoxy, 2-methylamino-1-methylethoxy, and 3-ethylaminopropoxy,
for di-[(1-6C)alkyl]amino-(2-6C)alkoxy: 2-dimethylaminoethoxy, 2-diethylaminoethoxy, 2-dimethylaminopropoxy, 2-dimethylamino-2-methylethoxy, 3-dimethylaminopropoxy and 4-dimethylaminobutoxy, 2-(N-methyl-N-isopropylamino)ethoxy, and 2-(N-ethyl-N-isopropylamino)ethoxy;
for (1-6C)alkoxy-(2-6C)alkoxy: 2-methoxyethoxy, 2-ethoxyethoxy, 3-methoxypropoxy, 2-methoxy-1-methylethoxy and 4-ethoxybutoxy;
for carbamoyl-(1-6C)alkoxy: carbamoylmethoxy and 2-carbamoylethoxy;
for N-(1-6C)alkylcarbamoyl-(1-6C)alkoxy: N-methylcarbamoylmethoxy, 2-(N-ethylcarbamoyl)ethoxy and 3-(N-methylcarbamoyl)propoxy;
for N,N-di-[(1-6C)alkyl]carbamoyl-(1-6C)alkoxy: N,N-dimethylcarbamoylmethoxy,
for amino-(1-6C)alkyl: aminomethyl, 2-aminoethyl, 1-aminoethyl and 3-aminopropyl;
for (1-6C)alkylamino-(1-6C)alkyl: methylaminomethyl, ethylaminomethyl, 1-methylaminoethyl, 2-methylaminoethyl;
for di-[(1-6C)alkyl]amino-(1-6C)alkyl: dimethylaminomethyl, diethylaminomethyl, dimethylaminoethyl, 2-dimethylaminoethyl and 3-dimethylaminopropyl;
for carbamoyl-(1-6C)alkyl: carbamoylmethyl, 1-carbamoylethyl, 2-carbamoylethyl and 3-carbamoylpropyl;
for N-(1-6C)alkylcarbamoyl-(1-6C)alkyl: N-methylcarbamoylmethyl, N-ethylcarbamoylmethyl, N-propylcarbamoylmethyl, 1-(1-methylcarbamoyl)ethyl, 1-(N-ethylcarbamoyl)ethyl, 2-(N-methylcarbamoyl)ethyl, 2-(N-ethylcarbamoyl)ethyl and 3-(N-methylcarbamoyl)propyl;
for N N-di-[(1-6C)alkyl]carbamoyl-(1-6C)alkyl: N,N-dimethylcarbamoylmethyl;
for hydroxy-(2-6C)alkylamino: 2-hydroxyethylamino, 3-hydroxypropylamino, 2-hydroxy-2-methylpropylamino and 4-hydroxybutylamino;
for cyano-(1-6C)alkylamino: cyanomethylamino, 2-cyanoethylamino and 3-cyanopropylamino;
for halogeno-(2-6C)alkylamino: 2-fluoroethylamino, 2-chloroethylamino, 2-bromoethylamino, 3-fluoropropylamino and 3-chloropropylamino;
for amino-(2-6C)alkylamino: 2-aminoethylamino, 3-aminopropylamino, 2-amino-2-methylpropylamino and 4-aminobutylamino;
for (1-6C)alkoxy-(2-6C)alkylamino: 2-methoxyethylamino, 2-ethoxyethylamino, 3-methoxypropylamino and 3-ethoxypropylamino;
for (1-6C)alkylamino-(2-6C)alkylamino: 2-methylaminoethylamino, 2-ethylaminoethylamino, 2-propylaminoethylamino, 3-methylaminopropylamino, 3-ethylaminopropylamino, 2-methylamino-2-methylpropylamino and 4-methylaminobutylamino;
for di-[(1-6C)alkyl]amino-(2-6C)alkylamino: 2-dimethylaminoethylamino, 2-(N-ethyl-N-methylamino)ethylamino, 2-diethylaminoethylamino, 2-dipropylaminoethylamino, 3-dimethylaminopropylamino, 3-diethylaminopropylamino, 2-dimethylamino-2-methylpropylamino and 4-dimethylaminobutylamino;
for N-(1-6C)alkylsulphamoyl: N-methylsulphamoyl and N-ethylsulphamoyl;
for N N-di-[(1-6C)alkyl]sulphamoyl: N,N-dimethylsulphamoyl;
for (1-6C)alkanesulphonylamino: methanesulphanamido and ethanesulphanamido;
for N-(1-6C)alkyl-(1-6C)alkanesulphonylamino: N-methylmethanesulphanamido and N-methylethanesulphanamido;
for (1-6C)alkanoylamino: formamido, acetamido, propionamido;
for N-(1-6C)alkyl-(1-6C)alkanoylamino: N-methylacetamido, and N-methylpropionamido;
for (1-6C)alkoxycarbonyl: methoxycarbonyl, ethoxycarbonyl, propoxycarbonyl and tert-butoxycarbonyl;
for (2-6C)alkanoyloxy: acetoxy and propionyloxy;
for heteroaryl-(1-6C)alkyl: heteroarylmethyl, heteroarylethyl, 2-heteroarylethyl, 2-heteroarylpropyl and 3-heteroarylpropyl;
for heteroaryl-(1-6C)alkoxy: heteroarylmethoxy and 2-heteroarylethoxy;
for heterocyclyl-(1-6C)alkyl: heterocyclylmethyl, 2-heterocyclylethyl, 2-heterocyclylpropyl and 3-heterocyclylpropyl;
for heterocyclyl-(1-6C)alkoxy: heterocyclylmethoxy, 2-heterocyclylethoxy, 3-heterocyclylpropoxy;
for aryl-(1-6C)alkyl: benzyl, 2-phenylethyl, 2-phenylpropyl and 3-phenylpropyl;
for aryl-(1-6C)alkoxy: benzyloxy and 2-phenylethoxy;
for aryloxy: phenoxy and 2-naphthyloxy;
for arylamino: anilino;
for (3-6C)cycloalkyl-(1-6C)alkyl: (3-6C)cycloalkylmethyl and (3-6C)cycloalkylethyl;
for (3-6C)cycloalkyl-(1-6C)alkoxy: (3-6C)cycloalkylmethoxy and (3-6C)cycloalkylethoxy;
for (1-6C)alkoxycarbonyl-(1-6C)alkyl: methoxycarbonylmethyl, ethoxycarbonylmethyl, tert-butoxycarbonylmethyl, 1-methoxycarbonylethyl, 1-ethoxycarbonylethyl, 2-methoxycarbonylethyl, 2-ethoxycarbonylethyl, 3-methoxycarbonylpropyl and 3-ethoxycarbonylpropyl;
for N-(1-6C)alkylcarbamoyl: N-methylcarbamoyl, N-ethylcarbamoyl and N-propylcarbamoyl;

for N,N-di-[(1-6C)alkyl]carbamoyl: N,N-dimethylcarbamoyl, N-ethyl-N-methylcarbamoyl and N,N-diethylcarbamoyl;
for halogeno-(1-6C)alkyl: fluoromethyl, chloromethyl, bromomethyl, difluoromethyl, dichloromethyl, trifluoromethyl, 2-fluoroethyl, 2-chloroethyl and 2-bromoethyl;
for hydroxy-(1-6C)alkyl: hydroxymethyl, 2-hydroxyethyl, 1-hydroxyethyl and 3-hydroxypropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for carboxy-(1-6C)alkyl: carboxymethyl, 1-carboxyethyl, 2-carboxyethyl, 3-carboxypropyl and 4-carboxybutyl;
for cyano-(1-6C)alkyl: cyanomethyl, 2-cyanoethyl, 1-cyanoethyl and 3-cyanopropyl;

A suitable pharmaceutically-acceptable salt of a compound of formula (I) is, for example, an acid-addition salt of a compound of formula (I) which is sufficiently basic, for example, an acid-addition salt with an inorganic or organic acid such as hydrochloric, hydrobromic, sulphuric, phosphoric, trifluoroacetic, citric, maleic, tartaric, fumaric, ascorbic, oleic, hemifumaric, succinic, hemisuccinic, mandelic, methanesulphonic, dimethanesulphonic, ethane-1,2-sulphonic, benzenesulphonic, salicylic or 4-toluenesulphonic acid.

Further values of m, R¹, R², R³, R⁴ and R⁵ are provided in the following embodiments of the invention. Such values may be used where appropriate with any of the definitions, claims or embodiments defined hereinbefore or hereinafter.

In an embodiment of the invention, m is 0 or 1.

In an embodiment of the invention, m is 1 or 2.

In an embodiment of the invention, m is 0

In an embodiment of the invention, m is 1.

In an embodiment of the invention, m is 2.

In an embodiment of the invention, R¹ is halogeno, hydroxy, cyano, trifluoromethyl, trifluoromethoxy, (1-6C)alkyl, (1-6C)alkoxy, (2-6C)alkenyl, (2-6C)alkynyl, (2-6C)alkanoyl, (1-6C)alkylthio, (1-6C)alkylsulphonyl, hydroxy-(2-6C)alkoxy, amino-(2-6C)alkoxy, cyano-(2-6C)alkoxy, (1-6C)alkylamino-(2-6C)alkoxy, di-[(1-6C)alkyl]amino-(2-6C)alkoxy, (1-6C)alkoxy-(2-6C)alkoxy, (1-6C)alkylamino-(2-6C)alkyl, di[(1-6C)alkyl]amino-(1-6C)alkyl, carbamoyl-(1-6C)alkyl, heteroaryl-(1-6C)alkyl, heteroaryl-(1-6C)alkoxy, heterocyclyl, heterocyclyl-(1-6C)alkyl, heterocyclyloxy and heterocyclyl-(1-6C)alkoxy,

and wherein any heteroaryl or heterocyclyl group in a R¹ substituent may optionally bear 1 or 2 substituents selected from hydroxy, halogeno, (1-6C)alkyl, (3-6C)cycloalkyl-(1-6C)alkyl, (3-6C)cycloalkyl-(1-6C)alkoxy, (1-6C)alkoxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylsulphonyl, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl,
and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, trifluoromethyl, oxo(1-6C)alkyl, (2-6C)alkenyl, (2-6C)alkynyl, (3-6C)cycloalkyl, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxycarbonyl, heteroaryl, heteroaryl-(1-6C)alkyl, heterocyclyl and heterocyclyloxy, and wherein any heterocyclyl group in a R¹ substituent may optionally bear 1 or 2 oxo or thioxo substituents.

It will be understood that where in the present specification it is stated that “any of the R¹ substituents defined hereinbefore which comprises a CH₂ group which is attached to 2 carbon atoms or a CH₃ group which is attached to a carbon atom may optionally bear on each said CH₂ or CH₃ group one or more substituents . . . ”, the substituents subsequently defined may be optionally present on any qualifying carbon atom in an R¹ group or on any qualifying carbon atom in an aforementioned substituent on an R¹ group.

In an embodiment of the invention, R¹ is heterocyclyl, heterocyclyloxy, heterocyclyl-(1-6C)alkoxy, (1-6C)alkoxy, (1-6C)alkylamino-(2-6C)alkoxy, di-[(1-6C)alkyl]amino-(2-6C)alkoxy, (1-6C)alkylamino-(2-6C)alkyl or di[(1-6C)alkyl]amino-(1-6C)alkyl,

and wherein any heterocyclyl group in a R¹ substituent may optionally bear 1 or 2 substituents selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl,
and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from hydroxy, amino, (1-6C)alkyl, (2-6C)alkenyl, (2-6C)alkynyl, (1-6C)alkoxy, (1-6C)alkylamino and di-[(1-6C)alkyl]amino.

In an embodiment of the invention, when R¹ is heterocyclyl, heterocyclyloxy or heterocyclyl-(1-6C)alkoxy, the heterocyclyl group may be selected from azetidinyl, piperidinyl, piperazinyl, 1,4-diazepanyl, pyrrolinyl, pyrrolidinyl, imidazolinyl, imidazolidinyl, pyrazolinyl, pyrazolidinyl and morpholinyl.
In an embodiment of the invention, R² is (1-6C)alkyl.
In an embodiment of the invention, R² is methyl.
In an embodiment of the invention, R³ is hydrogen, halogeno, trifluoromethyl, cyano or (1-6C)alkyl.
In an embodiment of the invention, R³ is hydrogen, halogeno or (1-6C)alkyl.
In an embodiment of the invention, R³ is hydrogen or halogeno. In an embodiment of the invention, R³ is hydrogen or fluoro.
In an embodiment of the invention, R³ is fluoro.
In an embodiment of the invention, R³ is hydrogen.
In an embodiment of the invention, R⁵ is hydrogen, halogeno, trifluoromethyl, cyano or (1-6C)alkyl.
In an embodiment of the invention, R⁵ is hydrogen, halogeno or (1-6C)alkyl.
In an embodiment of the invention, R⁵ is hydrogen or halogeno.
In an embodiment of the invention, R⁵ is hydrogen or fluoro.
In an embodiment of the invention, R⁵ is fluoro.
In an embodiment of the invention, R⁵ is hydrogen.
In an embodiment of the invention, R³ and R⁵ each represent hydrogen, halogeno or (1-6C)alkyl;
In an embodiment of the invention, R³ and R⁵ each represent is hydrogen or halogeno.
In an embodiment of the invention, R³ and R⁵ each represent hydrogen.

In an embodiment of the invention, R⁴ is aryl or heteroaryl, which aryl or heteroaryl is substituted with halogeno, (3-6C)cycloalkyl, heteroaryl or heterocyclyl, and which aryl or heteroaryl may further be optionally substituted by one or more substituents independently selected from halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, hydroxy(1-6C)alkylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphinoyl, heteroaryl-(1-06C)alkoxy, heteroarylamino, heterocyclyl, heterocyclyl-(1-6C)alkyl, heterocyclyloxy, heterocyclyl-(1-6C)alkoxy, heterocyclylamino, heterocyclylthio, aryl, aryl-(1-6C)alkyl, aryloxy, arylthio, arylthio, arylsulphinyl, aryl-(1-6C)alkoxy, arylamino, (3-6C)cycloalkyl, (3-6C)cycloalkyl-(1-6C)alkyl, (3-6C)cycloalkyloxy, (3-6C)cycloalkylthio, (3-6C)cycloalkyl-(1-6C)alkoxy and (3-6C)cycloalkylamino,

and wherein any heteroaryl, heterocyclyl, aryl or (3-6C)cycloalkyl group in a R⁴ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, (1-6C)alkylthio, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, (2-6C)alkanoyloxy, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl, and wherein any of the R⁴ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R⁴ substituent may optionally bear 1 or 2 oxo or thioxo substituents.

It will be understood that where in the present specification it is stated that “any of the R⁴ substituents defined hereinbefore which comprises a CH₂ group which is attached to 2 carbon atoms or a CH₃ group which is attached to a carbon atom may optionally bear on each said CH₂ or CH₃ group one or more substituents . . . ”, the substituents subsequently defined may be optionally present on any qualifying carbon atom in an R⁴ group or on any qualifying carbon atom in an aforementioned substituent on an R⁴ group.

In an embodiment of the invention, R⁴ is phenyl.

In an embodiment of the invention, when R⁴ is heteroaryl it is pyridyl.

In an embodiment of the invention R⁴ is aryl or heteroaryl, which aryl or heteroaryl is substituted with halogeno, (3-6C)cycloalkyl, or heterocyclyl; which (3-6C)cycloalkyl, heteroaryl or heterocyclyl may be optionally substituted by one or more substituents independently selected from halogeno, hydroxy, (1-6C)alkyl, (1-6C)alkoxy, (1-6C)alkylsulphinyl, trifluoromethyl, trifluoromethoxy; and wherein the aryl or heteroaryl R⁴ may further be optionally substituted by one or more substituents independently selected from halogeno, hydroxy, cyano, trifluoromethyl, trifluoromethoxy, amino, (1-6C)alkyl, (1-6C)alkoxy, (1-6C)alkylsulphinyl, (1-6C)alkylamino and di[(1-6C)alkyl]amino.

In an embodiment of the invention R⁴ is aryl or heteroaryl, which aryl or heteroaryl is substituted with (3-6C)cycloalkyl or heterocyclyl, which (3-6C)cycloalkyl or heterocyclyl may be optionally substituted by one or more substituents independently selected from halogeno, hydroxy, (1-6C)alkyl, (1-6C)alkoxy, (1-6C)alkylsulphinyl, trifluoromethyl, trifluoromethoxy; and wherein the aryl or heteroaryl R⁴ may further be optionally substituted by one or more substituents independently selected from halogeno, hydroxy, cyano, trifluoromethyl, trifluoromethoxy, amino, (1-6C)alkyl, (1-6C)alkoxy, (1-6C)alkylsulphinyl, (1-6C)alkylamino and di[(1-6C)alkyl]amino.

In an embodiment of the invention, when R⁴ is substituted with (3-6C)cycloalkyl, the (3-6C)cycloalkyl may be selected from cyclopropyl, cyclobutyl, cyclopentyl and cyclohexyl.

In an embodiment of the invention, when R⁴ is substituted with heterocyclyl, the heterocyclyl may be selected from azetidinyl, pyrrolinyl, pyrrolidinyl, imidazolinyl, imidazolidinyl, pyrazolinyl, pyrazolidinyl and morpholinyl.

In a further aspect of the invention there is provided a compound of formula (IB)

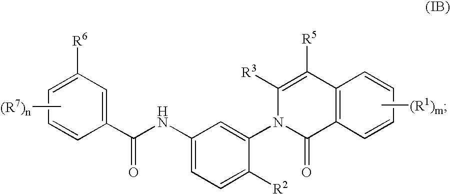

wherein
R⁶ is (3-6C)cycloalkyl or heterocyclyl, which (3-6C)cycloalkyl or heterocyclyl may be optionally substituted by one or more substituents independently selected from halogeno, hydroxy, (1-6C)alkyl, (1-6C)alkoxy, (1-6C)alkylsulphinyl, trifluoromethyl and trifluoromethoxy;
R⁷ is halogeno, hydroxy, cyano, trifluoromethyl, trifluoromethoxy, N,N-dialkylamino(1-6)alkylamino, N,N-dialkylamino(1-6)alkylthio, (1-6C)alkyl, (1-6C)alkoxy, (1-6C)alkylamino or di[(1-6C)alkyl]amino; and
n is 0, 1 or 2;
m is 0, 1 or 2;
R¹ is halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphonyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, aryl, aryl-(1-6C)alkyl, aryloxy, arylthio, arylsulphinyl, arylsulphonyl, aryl-(1-6C)alkoxy, arylamino, and wherein any aryl, heteroaryl or heterocyclyl group in a R¹ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl, and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)allyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R¹ substituent may optionally bear 1 or 2 oxo or thioxo substituents;
R² is halogeno or (1-6C)alkyl;
R³ is hydrogen, halogeno, trifluoromethyl, cyano or (1-6C)alkyl; and
R⁵ is hydrogen, halogeno, trifluoromethyl, cyano or (1-6C)alkyl;
or a pharmaceutically-acceptable salt thereof.

For compounds of formula (IB), embodiments of the invention include those wherein each of R¹, R², R³, R⁵ and m are as defined herein above in embodiments of the invention describing compounds of formula (I).

In a further aspect of the invention there is provided a compound of formula (IC)

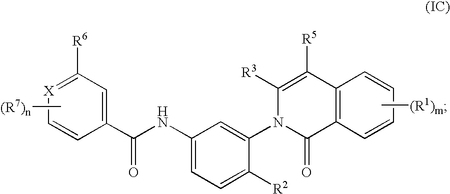

wherein

### X is CH or N,

R⁶ is halogeno, (3-6C)cycloalkyl, heteroaryl or heterocyclyl; which (3-6C)cycloalkyl, heteroaryl or heterocyclyl may be optionally substituted by one or more substituents independently selected from halogeno, hydroxy, (1-6C)alkyl, (1-6C)alkoxy, (1-6C)alkylsulphinyl, trifluoromethyl and trifluoromethoxy;
R⁷ is halogeno, hydroxy, cyano, trifluoromethyl, trifluoromethoxy, N,N-dialkylamino(1-6)alkylamino, N,N-dialkylamino(1-6)alkylthio, (1-6C)alkyl, (1-6C)alkoxy, (1-6C)alkylamino or di[(1-6C)alkyl]amino; and
n is 0, 1 or 2;
m is 0, 1 or 2;
R¹ is halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphonyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, aryl, aryl-(1-6C)alkyl, aryloxy, arylthio, arylsulphinyl, arylsulphonyl, aryl-(1-6C)alkoxy, arylamino, and wherein any aryl, heteroaryl or heterocyclyl group in a R¹ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl, and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R¹ substituent may optionally bear 1 or 2 oxo or thioxo substituents;
R² is halogeno or (1-6C)alkyl;
R³ is hydrogen, halogeno, trifluoromethyl, cyano or (1-6C)alkyl; and
R⁵ is hydrogen, halogeno, trifluoromethyl, cyano or (1-6C)alkyl;
or a pharmaceutically-acceptable salt thereof.

For compounds of formula (IC), embodiments of the invention include those wherein each of R¹, R², R³, R⁵ and m are as defined herein above in embodiments of the invention describing compounds of formula (I).

In an embodiment of the invention, for compounds of formula (IC), m is 1 and R¹ represents heterocyclyl, heterocyclyloxy, heterocyclyl-(1-6C)alkoxy, (1-6C)alkoxy, (1-6C)alkylamino-(2-6C)alkoxy, di-[(1-6C)alkyl]amino-(2-6C)alkoxy, (1-6C)alkylamino-(2-6C)alkyl or di[(1-6C)alkyl]amino-(1-6C)alkyl.

In another embodiment of the invention, for compounds of formula (IC), m is 1 and R¹ heterocyclyl-(1-6C)alkoxy, (1-6C)alkoxy, (1-6C)alkylamino-(2-6C)alkoxy or di-[(1-6C)alkyl]amino-(2-6C)alkoxy.

In an embodiment of the invention, for compounds of formula (IC) when R¹ is heterocyclyl, heterocyclyloxy or heterocyclyl-(1-6C)alkoxy, the heterocyclyl group may be selected from azetidinyl, piperidinyl, pyrrolinyl, pyrrolidinyl, imidazolinyl, imidazolidinyl, pyrazolinyl, pyrazolidinyl and morpholinyl.

In an embodiment of the invention, for compounds of formula (IC), R² is (1-6C)alkyl, e.g. methyl.
In an embodiment of the invention, for compounds of formula (IC), R³ is hydrogen.
In an embodiment of the invention, for compounds of formula (IC), R⁵ is hydrogen.
In an embodiment of the invention, for compounds of formula (IC), n is 0.
In an embodiment of the invention, for compounds of formula (IC), R⁶ is halogeno, heteroaryl or heterocyclyl, which heteroaryl or heterocyclyl may be optionally substituted by hydroxy.
In an embodiment of the invention, for compounds of formula (IC), R⁶ is heteroaryl, e.g. thienyl or oxazolyl.

In an embodiment of the invention, for compounds of formula (IC), R⁶ is heterocyclyl e.g. piperidinyl, pyrrolidinyl and morpholinyl.

In a further aspect of the invention there is provided a compound of formula (ID)

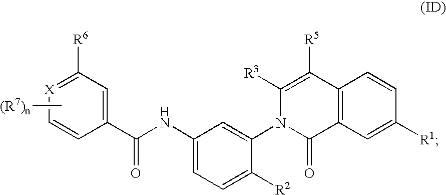

wherein R¹, R², R³, R⁴, R⁵, R⁶, R⁷, n and X are as defined herein above with respect to compounds of formula (I) or formula (IC), and embodiments of the invention include those wherein each of R¹, R², R³, R⁴, R⁵, R⁶, R⁷ n and X are as defined herein above in embodiments of the invention describing compounds of formula (IC).

In a further aspect the present invention provides a compound of formula (I) selected from:
N-{4-Methyl-3-[1-oxo-7-(2-pyrrolidin-1-ylethoxy)isoquinolin-2(1H)-yl]phenyl}-3-piperidin-1-ylbenzamide,
N-{4-Methyl-3-[1-oxo-7-(2-pyrrolidin-1-ylethoxy)isoquinolin-2(1H)-yl]phenyl}-3-pyrrolidin-1-ylbenzamide,
3-Azepan-1-yl-N-{4-methyl-3-[1-oxo-7-(2-pyrrolidin-1-ylethoxy)isoquinolin-2(1H)-yl]phenyl}benzamide,
3-[(3R)-3-Hydroxypyrrolidin-1-yl]-N-{4-methyl-3-[1-oxo-7-(2-pyrrolidin-1-ylethoxy)isoquinolin-2(1H)-yl]phenyl}benzamide,
N-{4-Methyl-3-[1-oxo-7-(2-pyrrolidin-1-ylethoxy)isoquinolin-2(1H)-yl]phenyl}-2-pyrrolidin-1-ylisonicotinamide,
N-{4-Methyl-3-[1-oxo-7-(2-pyrrolidin-1-ylethoxy)isoquinolin-2(1H)-yl]phenyl}-3-(tetrahydro-2H-pyran-4-yl)benzamide,
N-{4-methyl-3-[1-oxo-7-(3-pyrrolidin-1-ylpropoxy)isoquinolin-2(1H)-yl]phenyl}-3-(2-thienyl)benzamide,
N-{3-[7-[3-(dimethylamino)propoxy]-1-oxoisoquinolin-2(1H)-yl]-4-methylphenyl}-3-(2-thienyl)benzamide,
N-{3-[7-[2-(dimethylamino)ethoxy]-1-oxoisoquinolin-2(1H)-yl]-4-methylphenyl}-2-(2-thienyl)isonicotinamide,
N-{4-Methyl-3-[1-oxo-7-(2-pyrrolidin-1-ylethoxy)isoquinolin-2(1H)-yl]phenyl}-2-(2-thienyl)isonicotinamide, and
N-{4-Methyl-3-[1-oxo-7-(3-pyrrolidin-1-ylpropoxy)isoquinolin-2(1H)-yl]phenyl}-3-(1,3-oxazol-5-yl)benzamide,
   or a pharmaceutically-acceptable salt thereof.

Compounds of formula (I), or pharmaceutically-acceptable salts thereof, may be prepared by any process known to be applicable to the preparation of chemically-related compounds. Suitable processes are illustrated by, for example, those in WO 00/55153. Such processes, when used to prepare a novel compound of formula (I) are provided as a further feature of the invention and are illustrated by the following representative process variants in which, unless otherwise stated, R¹, R², R³, R⁴ and R⁵ have any of the meanings defined hereinbefore. Necessary starting materials may be obtained by standard procedures of organic chemistry. The preparation of some representative starting materials is described in conjunction with the following representative process variants and within the accompanying Examples. Alternatively necessary starting materials are obtainable by analogous procedures to those illustrated or are known in the literature and which are within the ordinary skill of an organic chemist.

Accordingly, the present invention further provides a process for preparing a compound of formula (I), or a pharmaceutically-acceptable sat thereof, which comprises

a) reacting a compound of formula (II) or a (1-6C)alkyl ester, acid anhydride or acid halide thereof, with a compound of formula (III)

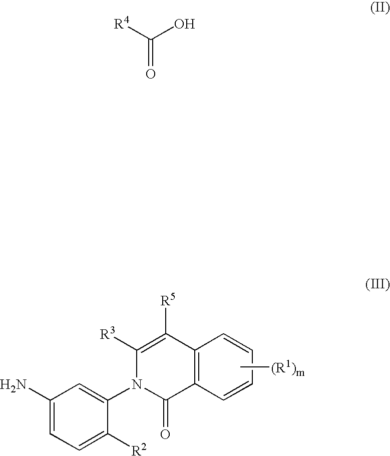

wherein R¹, m, R², R³, R⁴ and R⁵ are as defined in claim 1, or
b) dehydrating a compound of formula (XIII) wherein R¹, m, R², R³, R⁴ and R⁵ are as defined in claim 1

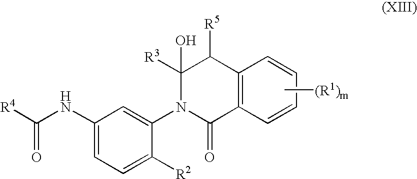

or
c) for compounds where m, R², R³, R⁴ and R⁵ are as defined in formula (I) and R¹ is amino-(2-6C)alkoxy, (1-6C)alkylamino-(2-6C)alkoxy, heterocyclyl-(2-6C)alkoxy or di-[(1-6C)alkyl]amino-(2,6C)alkoxy, reacting a compound of formula (XVIII), wherein L¹ represents a suitable leaving group and p is 1 to 5,

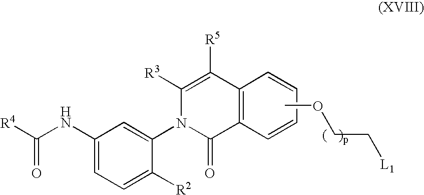

with an amine of formula HNWV wherein W and V are independently hydrogen, (1-6C)alkyl or, together with the nitrogen atom to which they are both attached, form a heterocyclyl ring that may optionally contain a further heteroatom, and optionally after a), b) or c) carrying out one or more of the following

(i) converting the compound to another compound of formula (I)

(ii) forming a pharmaceutically-acceptable salt of the compound.

In a) the reaction of (II) and (III) may be conveniently performed in an organic solvent such as acetone, dichloromethane, N,N-dimethylformamide, 1-methyl-2-pyrrolidinone, tetrahydrofuran, methylene chloride, 1,2-dimethoxyethane, N,N-dimethylacetamide, N-methylpyrrolidin-2-one, dimethylsulphoxide, and at a temperature in the range, for example, of from −78 to 150° C., conveniently at or near ambient temperature (20° C.). When compound (II) is in carboxylic acid form, it may be necessary or desirable to use a coupling agent such as bromo-tris-pyrrolidino-phosphonium hexafluorophosphate (PyBroP) at a non-extreme temperature in the reaction. If compound (II) is in acyl halide form, such compounds may be conveniently prepared by treatment of the corresponding carboxylic acid derivative under standard conditions (such as thionyl chloride or oxalyl chloride in dichloromethane with additional N,N-dimethylformamide) and used in a solvent such as acetone or dichloromethane with a suitable base such as potassium carbonate or triethylamine.

In b) the dehydration may be conducted by treating (XIII) with a suitable acid (for example, an inorganic or organic acid such as hydrochloric, hydrobromic, sulphuric, acetic, trifluoroacetic, citric or maleic acid) in a suitable inert solvent or diluent (for example water, methanol, ethanol, tetrahydrofuran, methylene chloride, 1,2-dimethoxyethane, N,N-dimethylformamide, N,N-dimethylacetamide, N-methylpyrrolidin-2-one, dimethylsulphoxide or acetone) and at a temperature in the range of from 0 to 150° C., conveniently at or near 25° C.

In c) (XVIII), wherein L₁ may for example represent chlorine, bromine, iodine or a suitably activated alcohol, may be reacted with an amine of formula HNWV in a suitable solvent, for example toluene, tetrahydrofuran, methylene chloride or ethanol at a temperature in the range of, for example 0° C. to 200° C., conveniently at or near 120° C. The reaction may be carried out in the presence of an inorganic or organic base such as sodium carbonate, potassium carbonate or a tertiary amine. The reaction is preferably carried out with an excess of the reacting amine or triethylamine in acetonitrile or ethanol at 120° C. (in a sealed vessel).

Compounds of formula (I) may be converted into other compounds of formula (I). For example compounds of formula (I) wherein R₄ is aryl or heteroaryl further substituted by halogen, may be converted, by coupling reaction, into other compounds of formula (I) wherein R₄ is aryl or heteroaryl further substituted by an aryl or heteroaryl. The coupling reaction is conveniently carried out in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C. Preferentially the reaction is carried out in the presence of potassium carbonate or sodium carbonate and tetrakis(triphenylphosphine)palladium(0) in a solvent such as tetrahydrofuran and water, or ethanol at a temperature of 60° C. to 120° C.

Compounds of formula (III) may be prepared from corresponding acids of formula (IV), or an acid derivative thereof, in a reaction such as a Curtius, Hoffman or Lossen rearrangement.

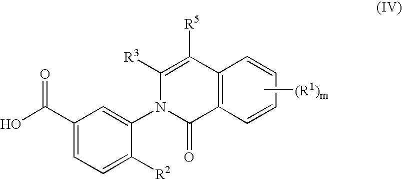

For example, under Curtius rearrangement conditions an acid of formula (IV) may be converted to (III) by reaction with diphenylphosphoryl azide in the presence of water or an appropriate alcohol (for example 2-methylpropanol, allyl alcohol, benzyl alcohol, 2-trimethylsilylethanol) with an organic amine base such as, for example, triethylamine or diisopropylethylamine. The reaction may conveniently be carried out in a suitable organic solvent or diluent, for example toluene, tetrahydrofuran, dioxane, dimethoxyethane, N,N-dimethylformamide, N,N-dimethylacetamide, N-methylpyrrolidin-2-one at elevated temperature, for example in the range of from 40 to 130° C.

Other conditions for converting (IV) to (III) include firstly activating the carboxyl group of the compound (IV), for example by treatment with a halo reagent (for example oxalyl or thionyl chloride) to form an acyl halide, which may for example be performed in an organic solvent at ambient temperature (e.g. at or near 20° C.), and then reacting the activated compound with an alkali metal azide followed by heating in the presence of an alcohol at elevated temperature, for example in the range of from 40 to 110° C.

Compounds of formula (II) are either commercially available, are known in the literature or may be prepared using known techniques.

Compounds of formula (IV) may be prepared by hydrolysis of a corresponding protected derivative of formula (V) wherein Z represents a protecting group (e.g. t-butoxy, benzyloxy, allyloxy, 9-fluorenylmethyloxy, cyclopropylamino) under basic conditions (for example by treating a 9-fluorenylmethyloxy derivative with piperidine), hydrogenolytic (for benzyloxy derivatives) or acidic conditions (for example by treating with 48% HBr or trifluoroacetic acid).

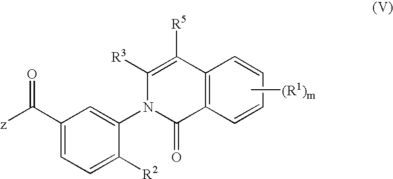

Compounds of formula (V) may be prepared by dehydration of a compound of formula (VI) by treatment with a suitable acid (for example, an inorganic or organic acid such as hydrochloric, hydrobromic, sulphuric, acetic, trifluoroacetic, citric or maleic acid) in a suitable inert solvent or diluent (for example water, methanol, ethanol, tetrahydrofuran, methylene chloride, 1,2-dimethoxyethane, N,N-dimethylformamide, N,N-dimethylacetamide, N-methylpyrrolidin-2-one, dimethylsulphoxide or acetone) and at a temperature in the range of from 0 to 150° C., conveniently at or near 25° C.

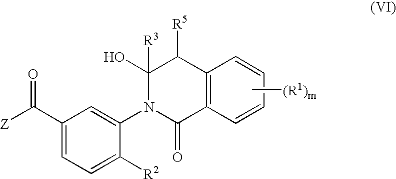

Compounds of formula (VI) may be prepared by reduction of the corresponding compound of formula (VII), with a suitable reducing agent, (for example, a metal hydride reducing agent such as sodium borohydride) in a suitable inert solvent or diluent, for example methanol, ethanol, tetrahydrofuran, methylene chloride, 1,2-dimethoxyethane, N,N-dimethylformamide, N,N-dimethylacetamide, N-methylpyrrolidin-2-one, dimethylsulphoxide or acetone, and at a temperature in the range of from 0 to 150° C., conveniently at or near 25° C.

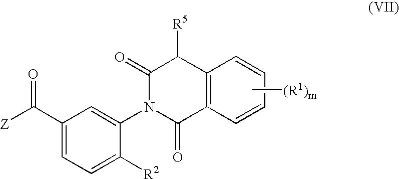

Compounds of formula (VII) may be prepared by reacting the corresponding compound of formula (VIII) with an aniline of formula (IX) wherein R¹, R², R⁵, m and Z are as defined hereinbefore and wherein any functional group is protected if necessary. The reaction may be performed in a suitable inert solvent or diluent, for example toluene, N,N-dimethylformamide, N,N-dimethylacetamide, N-methylpyrrolidin-2-one, dimethylsulphoxide or acetone, and at a temperature in the range, for example, of from 0 to 200° C., conveniently at or near 150° C.

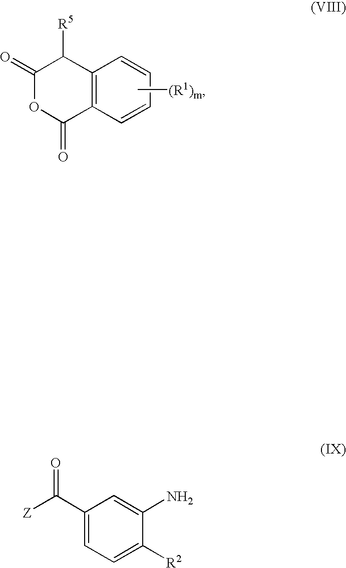

Compounds of formula (VIII) may be prepared by cyclisation of a reactive derivative of a compound of formula (X) wherein R¹ and R⁵ are as defined hereinbefore and wherein any functional group is protected if necessary.

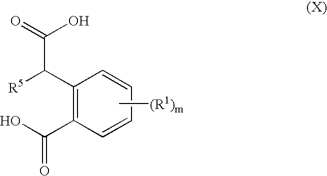

The cyclisation reaction may be carried out in a suitable inert solvent or diluent, for example toluene, N,N-dimethylformamide, N,N-dimethylacetamide, N-methylpyrrolidin-2-one, dimethylsulphoxide or acetone, and at a temperature in the range of from 0 to 200° C., conveniently at or near 25° C.

Suitable reactive derivatives of a compound of formula (X) are, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an anhydride formed by the reaction of the acid and acyl halide such as acetyl chloride; an active ester, for example an ester formed by the reaction of the acid with a phenol such as pentafluorophenol, with an ester such as pentafluorophenyl trifluoroacetate or with an alcohol such as N-hydroxybenzotriazole; an acyl azide, for example an azide formed by the reaction of the acid and an azide such as diphenylphosphoryl azide; an acyl cyanide, for example a cyanide formed by the reaction of an acid and a cyanide such as diethylphosphoryl cyanide; or the product of the reaction of the acid and a carbodiimide such as dicyclohexylcarbodiimide.

The cyclisation reaction may conveniently be carried out in the presence of a suitable base such as, for example, an alkali or alkaline earth metal carbonate, alkoxide, hydroxide or hydride, for example sodium carbonate, potassium carbonate, sodium ethoxide, potassium butoxide, sodium hydroxide, potassium hydroxide, sodium hydride or potassium hydride, or an organometallic base such as an alkyl-lithium, for example n-butyl-lithium, or a dialkylamino-lithium, for example lithium di-isopropylamide, or, for example, an organic amine base such as, for example, pyridine, 2,6-lutidine, collidine, 4-dimethylaminopyridine, triethylamine, morpholine or diazabicyclo[5.4.0]undec-7-ene.

The cyclisation reaction may also conveniently be carried out in the presence of a suitable acid such as, for example, an inorganic or organic acid such as hydrochloric, hydrobromic, sulphuric, acetic, trifluoroacetic, citric or maleic acid.

Compounds of formula (X) may be prepared by reaction of a 2-bromobenzoic acid of formula (XI) with a compound of formula (XII) wherein R⁵ is as defined hereinbefore, and wherein X is a suitable activated acetic acid equivalent.

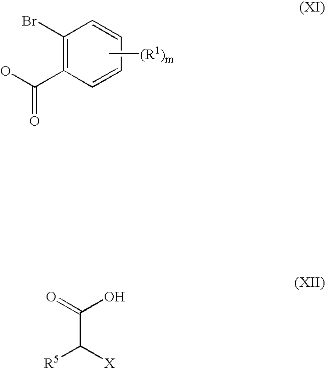

The reaction of (XI) and (XII) may conveniently be carried out in the presence of a suitable base such as, for example, an alkali or alkaline earth metal carbonate, alkoxide, hydroxide or hydride, for example sodium carbonate, potassium butoxide, or an organometallic base such as an alkyl-lithium, for example n-butyl-lithium, or a dialkylamino-lithium, for example lithium di-isopropylamide, or, for example, an organic amine base such as, for example, pyridine or 2,6-lutidine, at a temperature in the range of from 0 to 200° C., conveniently at or near 80° C. A suitable activated acetic acid equivalent of a compound of the formula (XII) is, for example, a protected malonic ester, for example dimethyl malonate; a β-keto ester, for example ethyl acetoacetate. The reaction of (XI) and (XII) may also involve the use of a suitable transition metal catalyst precursor, for example copper (I) bromide. The transformation may also be effected using the aryl iodides or aryl triflate versions of a compound of formula (XII).

Compounds of formula (XIII) (as used in process b) may be prepared according to Scheme 1.

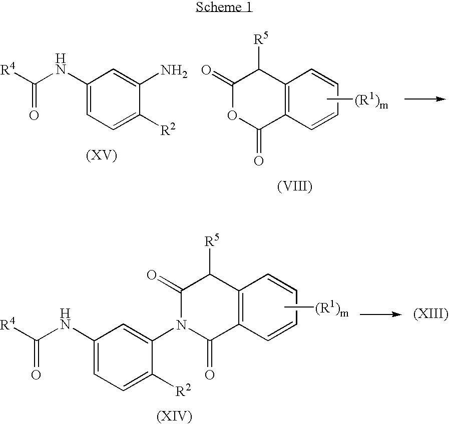

In scheme 1, compounds of formula (XIV) may be converted to compounds of formula (XIII) by employing conditions analogous to those previously described for the preparation of compounds of formula (VI). Compounds of formula (XIV) may be prepared by reacting the corresponding compound of formula (VIII) with a corresponding aniline of formula (XV) in conditions analogous to those previously described for the preparation of compounds of formula (VII).

Compounds of formula (XV), where R² and R⁴ are as defined in formula (I), may be prepared according to Scheme 2.

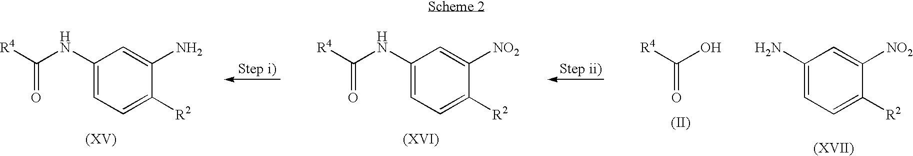

As depicted in Scheme 2 step i, compounds of formula (XV) may be prepared by reduction of the corresponding compounds of formula (XVI). Typical reaction conditions include the use of ammonium formate or hydrogen gas in the presence of a catalyst for example palladium-on-charcoal. Alternatively a dissolving metal reduction may be carried out for example using iron in the presence of an acid, for example an inorganic or organic acid such as hydrochloric acid, hydrobromic acid, sulphuric acid or acetic acid. The reaction is conveniently carried out in the presence of an organic solvent such as methanol, ethanol, propan-2-ol or water and at a temperature between 25° C. and 200° C. The reaction is preferably carried out using iron and ammonium chloride in a polar protic solvent, preferably a mixture of ethanol and water, and preferably with heating for example to between 60° C. and 90° C.

As depicted in scheme 2 step ii, compounds of formula (XVI) may be prepared by reacting a compound of formula (II) or a (1-6C)alkyl ester, acid anhydride or acid halide thereof, with a compound of formula (XVII) by employing conditions as previously described for the preparation of compounds of formula (I) by reacting a compound of formula (II) with a compound of formula (III).

Compounds of formula (XVIII) (as used in process c) may be prepared according to

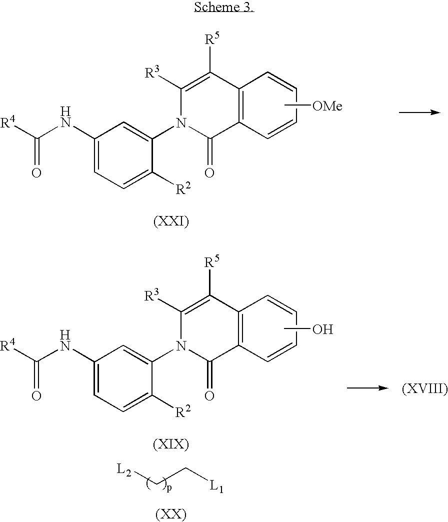

As depicted in Scheme 3, compounds of formula (XVIII) may be prepared by alkylating a compound of formula (XIX) with a suitable alkylating agent such as (XX) wherein L₁ and L₂ are independently suitable leaving groups such as chlorine, bromine, iodine or a suitably activated alcohol and p is 1 to 5. The reaction may be performed in an inert organic solvent such as dichloromethane, N,N-dimethylformamide, tetrahydrofuran, 1,4-dioxane or acetonitrile and in the presence of an inorganic or organic base such as potassium carbonate, sodium carbonate, triethylamine or N,N-diisopropylethylamine. For example the reaction may be performed at a temperature range between 40° C. and 150° C. Preferably the reaction is carried out using N,N-dimethylformamide or acetonitrile and potassium carbonate between 60° C. and 90° C.

Compounds of formula (XIX) may be prepared by dealkylation of compounds of formula (XXI). The reaction may be performed by employing Lewis acids such as boron tribromide or boron trifluoride. Preferably nucleophilic dealkylation conditions are used employing reagents such as lithium iodide or sodium ethanethiolate. The reaction may be performed in an organic solvent such as acetonitrile, dimethylsulphoxide, N-methylpyrrolidin-2-one, N,N-dimethylacetamide, dimethoxyethane or 1,4-dioxane at temperatures in the range 25° C. to 300° C. Preferably the reaction is carried out using N,N-dimethylformamide or 2,4,6-collidine at between 140° C. and 200° C.

Compounds of formula (XXI) may be prepared as described in scheme 1. Compounds of formula (II), (IX), (XI), (XII) and (XVII) are either commercially available, are known in the literature or may be prepared using known techniques.

It will be appreciated by those skilled in the art that in the processes of the present invention certain functional groups such as hydroxy, carboxy or amino groups in the starting reagents or intermediate compounds may need to be protected by protecting groups. Thus, the preparation of the compounds of formula (I) may involve at a certain stage protection with and/or the removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994). The compounds of formula (I) above may be converted to a pharmaceutically acceptable salt using conventional methods.

The compounds of the invention have activity as pharmaceuticals, in particular as p38 kinase inhibitors. Diseases and conditions which may be treated with the compounds include:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory is myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further aspect of the invention there is provided a pharmaceutical composition which comprises a compound of formula (I), or a pharmaceutically-acceptable salt thereof, in association with a pharmaceutically-acceptable diluent or carrier.

According to a further aspect of the invention there is provided a pharmaceutical composition for use in the treatment of diseases mediated by cytokines which comprises a compound of formula (I), or a pharmaceutically-acceptable salt thereof, in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder), for parenteral administration (for example as a sterile aqueous or oily solution for intravenous, subcutaneous, intramuscular or intramuscular dosing or as a suppository for rectal dosing) or for intra-articular administration. The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active, agent compounded with an appropriate and convenient amount of excipients which may vary from about 5 to about 98 percent by weight of the total composition. Alternatively, a formulation intended for inhaled administration to humans will generally contain, for example, from 0.5 μg to 5 mg of active agent compounded with an appropriate and convenient amount of excipients which may vary from about 10 to about 90 percent by weight of the total composition.

The size of the dose for therapeutic or prophylactic purposes of a compound of formula (I) of the invention will naturally vary according to the nature and severity of the conditions, the age and sex of the animal or patient and the route of administration, according to well known principles of medicine.

In using a compound of formula (I) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used.

A particular aspect of the present invention relates to pharmaceutical compositions that are formulated to allow the compounds described herein to be administered locally to the lung. Advantages associated with such inhaled drug delivery include large lung surface area for dose absorption; rapid drug absorption, rapid onset of action; avoidance of the gastrointestinal tract and first-pass metabolism, lower dose and reduced side effects. Administration to the lung is of particular benefit when the compounds are used to treat respiratory diseases such as asthma or chronic obstructive pulmonary disease. For administration to the lung, a dose in the range, for example, of 0.5 μg to 25 mg per kg body weight may be used.

According to a further aspect of the invention there is provided a compound of formula (I), or a pharmaceutically-acceptable salt thereof, for use in a method of treatment of the human or animal body by therapy.

According to a further aspect of the invention there is provided the use of a compound of formula (I), or a pharmaceutically-acceptable salt thereof, in the manufacture of a medicament.

According to a further aspect of the invention there is provided the use of a compound of formula (I), or a pharmaceutically-acceptable salt thereof, in the manufacture of a medicament for use in the treatment of medical conditions mediated by cytokines.

In a further aspect the present invention provides a method of treating diseases or medical conditions mediated by cytokines which comprises administering to a warm-blooded animal an effective amount of a compound of formula (I), or a pharmaceutically-acceptable salt thereof.

In a further aspect the present invention provides a method of treating a disease or medical condition mediated by cytokines which comprises administering to a warm-blooded animal in need thereof a cytokine inhibiting amount of a compound of formula (I), or a pharmaceutically-acceptable salt thereof.

In a further aspect the present invention provides a method of treating a disease or medical condition mediated by the production or effect of cytokines which comprises administering to a warm-blooded animal in need thereof a cytokine inhibiting amount of a compound of formula (I), or a pharmaceutically-acceptable salt thereof.

In a further aspect on the invention there is provided a method for inhibiting the production or effect of a cytokine in a warm-blooded animal in need thereof a p38 kinase inhibiting amount of a compound of formula (I), or a pharmaceutically-acceptable salt thereof.

In a further aspect the present invention provides the use of a compound of formula (I), or a pharmaceutically-acceptable salt thereof, in the manufacture of a medicament for use in the treatment of diseases or medical conditions mediated by TNF, IL-1, IL-6 or IL-8.

In a further aspect the present invention provides a method of treating diseases or medical conditions mediated by TNF, IL-1, IL-6 or IL-8 which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable salt thereof in the manufacture of a medicament for use in the treatment of diseases or medical conditions mediated by TNF.

In a further aspect the present invention provides a method of treating diseases or medical conditions mediated by TNF which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable salt thereof, in the manufacture of a medicament for use in inhibiting TNF, IL-1, IL-6 or IL-8.

In a further aspect the present invention provides a method of inhibiting TNF, IL-1, IL-6 or IL-8 which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable salt thereof, in the manufacture of a medicament for use in inhibiting TNF.

In a further aspect the present invention provides a method of inhibiting TNF which comprises administering to a warm-blooded animal an effective amount of a compound of formula (I), or a pharmaceutically-acceptable salt thereof.

In a further aspect the present invention provides a compound of formula (I), or a pharmaceutically-acceptable salt thereof, in the manufacture of a medicament for use in the treatment of diseases or medical conditions mediated by p38 kinase.

In a further aspect the present invention provides a method of treating diseases or medical conditions mediated by p38 kinase which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable salt thereof, in the manufacture of a medicament for use in the production of a p38 kinase inhibitory effect.

In a further aspect the present invention provides a method of providing a p38 kinase inhibitory effect which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable thereof, in the manufacture of a medicament for use in the treatment of rheumatoid arthritis, asthma, chronic obstructive pulmonary disease, inflammatory bowel disease, multiple sclerosis, AIDS, septic shock, congestive heart failure, ischaemic heart disease or psoriasis.

In a further aspect the present invention provides a method of treating rheumatoid arthritis, asthma, chronic obstructive pulmonary disease, inflammatory bowel disease, multiple sclerosis, AIDS, septic shock, congestive heart failure, ischaemic heart disease or psoriasis which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable thereof, in the manufacture of a medicament for use in the treatment of a respiratory disease such as asthma, chronic obstructive pulmonary disease or rhinitis.

In a further aspect the present invention provides a method of treating a respiratory disease such as asthma, chronic obstructive pulmonary disease or rhinitis which comprises administering to a warm-blooded animal an effective amount of a compound of formula (I), or a pharmaceutically-acceptable salt thereof.

In a further aspect the present invention provides the use of a compound of formula (I), or a pharmaceutically-acceptable thereof, in the manufacture of a medicament for use in the treatment of chronic obstructive pulmonary disease.

In a further aspect the present invention provides a method of treating chronic obstructive pulmonary disease which comprises administering to a warm-blooded animal an effective amount of a compound of formula (I), or a pharmaceutically-acceptable salt thereof.

A compound of formula (I), may be used in combination with other drugs and therapies used in the treatment of disease states which would benefit from the inhibition of cytokines, in particular TNF and IL-1. For example, a compound of formula (I), could be used in combination with drugs and therapies used in the treatment of rheumatoid arthritis, asthma, chronic obstructive pulmonary disease, inflammatory bowel disease, multiple sclerosis, AIDS, septic shock, congestive heart failure, ischaemic heart disease, psoriasis and the other disease states mentioned earlier in this specification.

For example, by virtue of its ability to inhibit cytokines, a compound of formula (I), is of value in the treatment of certain inflammatory and non-inflammatory diseases which are currently treated with a cyclooxygenase-inhibitory non-steroidal anti-inflammatory drug (NSAID) such as indomethacin, ketorolac, acetylsalicyclic acid, ibuprofen, sulindac, tolmetin and piroxicam. Co-administration of a compound of formula (I) of the present invention with a NSAID can result in a reduction of the quantity of the latter agent needed to produce a therapeutic effect. Thereby the likelihood of adverse side-effects from the NSAID such as gastrointestinal effects are reduced. Thus according to a further feature of the invention there is provided a pharmaceutical composition which comprises a compound of formula (I), or a pharmaceutically-acceptable salt thereof, in conjunction or admixture with a cyclooxygenase inhibitory non-steroidal anti-inflammatory agent, and a pharmaceutically-acceptable diluent or carrier.

A compound of formula (I), may also be used with anti-inflammatory agents such as an inhibitor of the enzyme 5-lipoxygenase.

A compound of formula (I) may also be used in the treatment of conditions such as rheumatoid arthritis in combination with antiarthritic agents such as gold, methotrexate, steroids and pencillinamine, and in conditions such as osteoarthritis in combination with steroids.

A compound of formula (I) may also be administered in degradative diseases, for example osteoarthritis, with chondroprotective, anti-degradative and/or reparative agents such as Diacerhein, hyaluronic acid formulations such as Hyalan, Rumalon, Arteparon and glucosamine salts such as Antril.

A compound of formula (I) may be used in the treatment of asthma in combination with antiasthmatic agents such as steroids, bronchodilators and leukotriene antagonists.

In particular, for the treatment of the inflammatory diseases rheumatoid arthritis, psoriasis, inflammatory bowel disease, chronic obstructive pulmonary disease, asthma and allergic rhinitis a compound of the present invention may be combined with agents such as TNF-α inhibitors such as anti-TNF monoclonal antibodies (such as Remicade, CDP-870 and D₂E₇) and TNF receptor immunoglobulin molecules (such as Enbrel®), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, leflunomide; ciclesonide; hydroxychloroquine, d-penicillamine, auranofin or parenteral or oral gold.

The present invention still further relates to the combination of a compound of formula (I) together with a leukotriene biosynthesis inhibitor, 5-lipoxygenase (5-LO) inhibitor or 5-lipoxygenase activating protein (FLAP) antagonist such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of formula (I) together with a receptor antagonist for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of formula (I) together with a PDE4 inhibitor including inhibitors of the isoform PDE4D.

The present invention still further relates to the combination of a compound of formula (I) together with a antihistaminic H₁ receptor antagonists such as cetirizine, loratadine, desloratadine, fexofenadine, astemizole, azelastine, and chlorpheniramine.

The present invention still further relates to the combination of a compound of formula (I) together with a gastroprotective H₂ receptor antagonist.

The present invention still further relates to the combination of a compound of formula (I) together with an α₁- and α₂-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of formula (I) together with anticholinergic agents such as ipratropium bromide; tiotropium bromide; oxitropium bromide; pirenzepine; and telenzepine.

The present invention still further relates to the combination of a compound of formula (I) together with a β₁- to β₄-adrenoceptor agonists such as metaproterenol, isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of formula (I) together with an insulin-like growth factor type I (IGF-1) mimetic.

The present invention still further relates to the combination of a compound of formula (I) together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of formula (I) together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of formula (I) together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to the combination of a compound of formula (I) together with antiviral agents such as Viracept, AZT, aciclovir and famciclovir, and antisepsis compounds such as Valant.

The present invention still further relates to the combination of a compound of formula (I) together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of formula (I) together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of formula (I) together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinin-B₁- and B₂-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK₁ and NK₃ receptor antagonists selected from the group consisting of NKP-608C; SB-233412 (talnetant); and D-4418; (xx) elastase inhibitors selected from the group consisting of UT-77 and ZD-0892; (xxi) TNF? converting enzyme inhibitors (TACE); (xxii) induced nitric oxide synthase inhibitors (iNOS) or (xxiii) chemoattractant receptor-homologous molecule expressed on TH2 cells, (CRTH2 antagonists).

A compound of formula (I) may also be used in combination with osteoporosis agents such as roloxifene, droloxifene, lasofoxifene or fosomax and immunosuppressant agents such as FK-506, rapamycin, cyclosporine, azathioprine, and methotrexate. A compound of formula (I) may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

A compound of formula (I) can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

If formulated as a fixed dose such combination products employ a compound of formula (I) within the dosage range described herein and the other pharmaceutically-active agent within its approved dosage range. Sequential use is contemplated when a combination formulation is inappropriate.

In a further embodiment the present invention provides a pharmaceutical product comprising, in combination, a first active ingredient which is a compound of formula (I), or a pharmaceutically acceptable salt thereof, as hereinbefore described, and at least one further active ingredient selected from:—
a phosphodiesterase inhibitor
a β2. adrenoceptor agonist
a modulator of chemokine receptor function
a protease inhibitor
a steroidal glucocorticoid receptor agonist
an anticholinergic agent, and a
a non-steroidal glucocorticoid receptor agonist.

The pharmaceutical product according to this embodiment may, for example, be a pharmaceutical composition comprising the first and further active ingredients in admixture. Alternatively, the pharmaceutical product may, for example, comprise the first and further active ingredients in separate pharmaceutical preparations suitable for simultaneous, sequential or separate administration to a patient in need thereof. The pharmaceutical product of this embodiment is of particular use in treating respiratory diseases such as asthma, COPD or rhinitis.

Examples of a phosphodiesterase inhibitor that may be used in the pharmaceutical product according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast),
8-amino-1,3-bis(cyclopropylmethyl)xanthine (Cipamfylline),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-yl methanesulphonate (Lirimilast),
(−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine),
3-(cyclopropylmethoxy)-N-(3,5-dichloro-4-pyridyl)-4-(difluoromethoxy)benzamide (Roflumilast),
the N-oxide of Roflumilast,
5,6-diethoxybenzo[b]thiophene-2-carboxylic acid (Tibenelast),
2,3,6,7-tetrahydro-2-(mesitylimino)-9,10-dimethoxy-3-methyl-4H-pyrimido[6,1-a]isoquinolin-4-one (trequinsin) and
3-[[3-(cyclopentyloxy)-4-methoxyphenyl]-methyl]-N-ethyl-8-(1-methylethyl)-3H-purine-6-amine (V-11294A).

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonists, for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921, in US 2005/222144, compounds GSK 159797, GSK 159802, GSK 597901, GSK 642444 and GSK 678007.

Examples of a modulator of chemokine receptor function that may be used in the pharmaceutical product according to this embodiment include a CCR1 receptor antagonist.

Examples of a protease inhibitor that may be used in the pharmaceutical product according to this embodiment include an inhibitor of neutrophil elastase or an inhibitor of MMP12.

Examples of a steroidal glucocorticoid receptor agonist that may be used in the pharmaceutical product according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3 S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of an anticholinergic agent that may be used in the pharmaceutical product according to this embodiment include for example a muscarinic receptor antagonist (for example a M1, M2 or M3 antagonist, such as a M3 antagonist) for example ipratropium (e.g. as bromide), tiotropium (e.g. as bromide), oxitropium (e.g. as bromide), tolterodine, pirenzepine, telenzepine, glycopyrronium bromide (such as R,R-glycopyrronium bromide or a mixture of R,S- and S,R-glycopyrronium bromide); mepensolate (e.g. as bromide), a quinuclidine derivative such as 3(R)-(2-hydroxy-2,2-dithien-2-ylacetoxy)-1-(3-phenoxypropyl)-1-azonia-bicyclo[2.2.2]octane bromide as disclosed in US 2003/0055080, quinuclidine derivatives as disclosed in WO 2003/087096 and WO 2005/115467 and DE 10050995; or GSK 656398 or GSK 961081.

Examples of a modulator of a non-steroidal glucocorticoid receptor agonist that may be used in the pharmaceutical product according to this embodiment include those described in WO2006/046916.

The invention is illustrated by the following non-limiting examples.

In the examples the NMR spectra were measured on a Varian Unity Inova spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column or Phenomenex Gemini® using 0.1% aqueous trifluoroacetic acid: acetonitrile, 0.1% aqueous ammonia: acetonitrile or 0.1% ammonium acetate: acetonitrile as the eluent. SCX and NH₂ resin were obtained from Varian Incorporated. Reactions that were heated by microwave irradiation were performed using either a Personal Chemistry Emrys Optimizer or a CEM Discover Microwave. Compound names were generated using the commercially available IUPAC chemical naming software package ACDLABS 8.0.

### EXAMPLE 1

### N-{4-Methyl-3-[1-oxo-7-(2-pyrrolidin-1-ylethoxy)isoquinolin-2(1H)-yl]phenyl}-3-piperidin-1-ylbenzamide

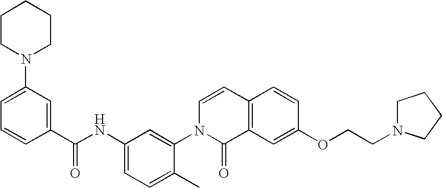

### a) N-Cyclopropyl-4-methyl-3-nitrobenzamide

To a stirred solution of 4-methyl-3-nitrobenzoyl chloride (20 g) in methylene chloride (200 ml) at 0° C. was added a mixture of cyclopropylamine (7.62 ml) and triethylamine (28 ml). The mixture was allowed to warm to room temperature and stirred for a further 16 hours. The reaction mixture was evaporated in vacuo and a saturated sodium bicarbonate solution was added. The precipitated solid was collected by filtration and washed with iso-hexane to give N-cyclopropyl-4-methyl-3-nitrobenzamide as a colourless solid (22.9 g). NMR Spectrum: (DMSO-d₆) 8.67 (d, 1H), 8.41 (d, 1H), 8.06 (m, 1H), 7.60 (d, 1H), 2.87 (m, 1H), 2.56 (s, 3H), 0.72 (m, 2H), 0.60 (m, 2H); Mass Spectrum: M+H⁺ 221.

### b) 3-Amino-N-cyclopropyl-4-methylbenzamide

A suspension of the product of step a) (22.9 g) and 10% palladium on carbon (2 g) in ethanol (500 ml) was agitated under a hydrogen atmosphere for 16 hours. The reaction mixture was filtered through diatomaceous earth (Celite®) and the filtrate concentrated to dryness to give 3-amino-N-cyclopropyl-4-methylbenzamide as a colourless solid (17.1 g). NMR Spectrum: (DMSO-d₆) 8.09 (d, 1H), 7.06 (d, 1H), 6.92 (m, 2H), 2.80 (m, 1H), 2.07 (s, 3H), 0.65 (m, 2H), 0.53 (m, 2H); Mass Spectrum: M+H⁺191.

### c) 7-Methoxy-1H-isochromene-1,3(4H)-dione

To a suspension of 2-(carboxymethyl)-5-methoxybenzoic acid (5.22 g) (synthesised using the procedure in Tetrahedron 1975, 31, 2607-19) in acetone (50 ml) was added acetyl chloride (7.06 ml) and the reaction mixture was stirred at room temperature for 18 hours. The solvent was evaporated and azeotroped with toluene (x3). The resultant solid was triturated with diethyl ether to yield 7-methoxy-1H-isochromene-1,3(4H)-dione as a brown solid (4.36 g). NMR Spectrum: (DMSO-d₆) 7.50 (s, 1H), 7.36 (m, 2H), 4.20 (s, 214), 3.84 (s, 3H).

### d) N-Cyclopropyl-3-(7-methoxy-1,3-dioxo-3,4-dihydroisoquinolin-2(1H)-yl)-4-methylbenzamide

A suspension of the product of step c) (1.09 g) and the product of step b) (1.19 g) in a mixture of toluene (9 ml) and acetic acid (3 ml) was heated under microwave irradiation conditions (Personal Chemistry Emrys Optimizer with 300W magnetron) at 150° C. for 90 minutes. This was repeated on three further batches and the separate batches combined, diluted with ethyl acetate and extracted with 2N HCl, water, brine, dried (magnesium sulfate) and left to crystallise for 18 hours. The solid was collected by filtration, washed with diethyl ether and air dried to yield N-cyclopropyl-3-(7-methoxy-1,3-dioxo-3,4-dihydroisoquinolin-2(1H)-yl)-4-methylbenzamide as a colourless solid (6.27 g); NMR Spectrum: (DMSO-d₆) 8.40 (d, 1H), 7.81 (d, 1H), 7.65 (s, 1H), 7.53 (s, 1H), 7.43 (m, 2H), 7.33 (d, 1H), 4.32 (d, 1H), 4.23 (d, 1H), 3.84 (s, 3H), 2.85 (m, 1H), 2.09 (s, 3H), 0.69 (m, 2H), 0.57 (m, 2H); Mass Spectrum: M+Na⁺ 387.

### e) N-Cyclopropyl-3-(7-methoxy-1-oxoisoquinolin-2(1H)-yl)-4-methylbenzamide

To a solution of the product of step d) (1 g) in methanol (20 ml) and methylene chloride (45 ml) under an atmosphere of argon was added sodium borohydride (114 mg) portionwise and the reaction stirred at room temperature for 17 hours. Concentrated hydrochloric acid (0.2 ml) was added and the reaction stirred for a further 4 hours. The reaction mixture was concentrated and the resultant solid triturated with ethyl acetate and air dried to yield N-cyclopropyl-3-(7-methoxy-1-oxoisoquinolin-2(1H)-yl)-4-methylbenzamide as a colourless solid (849 mg); NMR Spectrum: (DMSO-d₆) 8.44 (d, 1H), 7.89 (d, 1H), 7.71 (d, 1H), 7.67 (s, 1H), 7.50 (d, 1H), 7.41 (d, 1H), 7.21 (d, 1H), 6.72 (d, 1H), 3.39 (s, 3H), 2.86 (m, 1H), 2.10 (s, 3H), 0.69 (m, 2H), 0.56 (m, 2H); Mass Spectrum: M+H⁺ 349.

### f) N-Cyclopropyl-3-(7-hydroxy-1-oxoisoquinolin-2(1H)-yl)-4-methylbenzamide

A stirred suspension of the product of step e) (845 mg) and lithium iodide (585 mg) in 2,4,6-collidine (10 ml) was heated under microwave irradiation conditions (Personal Chemistry Emmys Optimizer with 300W magnetron) at 200° C. for 90 minutes. The mixture was dissolved using 2N aqueous sodium hydroxide and re-acidified using 2N aqueous hydrochloric acid. The aqueous phase was extracted with ethyl acetate (x4) and the combined organic layers concentrated. The residue was triturated with 2N aqueous hydrochloric acid and the solid collected by filtration, washed with diethyl ether and air dried to yield N-cyclopropyl-3-(7-hydroxy-1-oxoisoquinolin-2(1H)-yl)-4-methylbenzamide as a brown solid (562 mg); NMR Spectrum: (DMSO-d₆) 10.00 (s, 1H), 8.43 (d, 1H), 7.88 (d, 1H), 7.73 (s, 1H), 7.61 (d, 1H), 7.60 (s, 1H), 7.49 (d, 1H), 7.24 (d, 1H), 7.11 (d, 1H), 6.66 (d, 1H), 2.85 (m, 1H), 2.10 (s, 3H), 0.69 (m, 2H), 0.55 (m, 2H); Mass Spectrum: M+Na⁺ 357.

### g) 3-(7-Hydroxy-1-oxoisoquinolin-2(1H)-yl)-4-methylbenzoic acid

The product of step f) (0.5 g) was stirred in 48% hydrobromic acid (7 mL) and heated under microwave irradiation conditions (Personal Chemistry Emrys Optimizer with 300W magnetron) at 150° C. for 1 hour. The reaction mixture was diluted with ethyl acetate, washed with water (x3), brine, dried over magnesium sulfate, filtered and concentrated to a brown solid. The solid was triturated with diethyl ether to yield 3-(7-hydroxy-1-oxoisoquinolin-2(1H)-yl)-4-methylbenzoic acid as a colourless solid (0.32 g); NMR Spectrum: (DMSO-d₆) 13.05 (s, 1H), 10.09 (s, 1H), 7.95 (d, 1H), 7.78 (s, 1H), 7.61 (d, 1H), 7.60 (s, 1H), 7.54 (d, 1H), 7.27 (d, 1H), 7.12 (d, 1H), 6.65 (d, 1H), 2.11 (s, 3H); Mass Spectrum: M+H⁺ 296.

### h) 2-Pyrrolidin-1-ylethyl 4-methyl-3-[1-oxo-7-(2-pyrrolidin-1-ylethoxy)isoquinolin-2(1H)-yl]benzoate

A mixture of the product of step g) (0.51 g), N-(2-chloroethyl)pyrrolidine hydrochloride (0.64 g), potassium carbonate (0.95 g) and tetrabutylammonium iodide (1.39 g) in N,N-dimethylformamide (15 ml) was heated under nitrogen at 60° C. for 12 hours. The reaction mixture was then partitioned between ethyl acetate and water. The organic layer was separated, washed with water and then dried over magnesium sulphate, filtered and concentrated to dryness in vacuo. The residue was purified by SiO₂ chromotography, eluting with 1% triethylamine and 5% methanol in dichloromethane to afford 2-pyrrolidin-1-ylethyl 4-methyl-3-[1-oxo-7-(2-pyrrolidin-1-ylethoxy)isoquinolin-2(1H)-yl]benzoate as a colourless gum (0.90 g). NMR Spectrum: (CDCl₃) 8.03 (d, 1H), 7.93 (s, 1H), 7.87 (s, 1H), 7.51 (d, 1H), 7.43 (d, 1H), 7.36 (d, 1H), 6.91 (d, 1H), 6.56 (d, 1H), 4.45 (t, 2H), 4.25 (t, 2H), 2.95 (t, 2H), 2.85 (t, 2H), 2.64-2.55 (m, 8H), 2.23 (s, 3H), 1.82-1.79 (m, 8H); Mass Spectrum: M+H⁺ 490

### i) 4-Methyl-3-[1-oxo-7-(2-pyrrolidin-1-ylethoxy)isoquinolin-2(1H)-yl]benzoic acid

A solution of the product from step h) (0.90 g) in methanol (20 ml) was treated with a solution of lithium hydroxide monohydrate (0.15 g) in water (10 ml). The mixture was stirred at room temperature for 18 hours. The methanol was removed in vacuo and the remaining aqueous adjusted to pH7 by the dropwise addition of acetic acid. The solution was cooled and the resulting precipitate collected by filtration and washed with water then acetonitrile to give 4-methyl-3-[1-oxo-7-(2-pyrrolidin-1-ylethoxy)isoquinolin-2(1H)-yl]benzoic acid as a colourless solid (0.40 g). NMR Spectrum: (D₂O, NaOD) 7.87 (d, 1H), 7.71 (s, 1H), 7.65-7.63 (m, 2H), 7.45 (d, 1H), 7.40-7.37 (m, 1H), 7.07 (d, 1H), 6.82 (d, 1H), 4.18 (s, 2H), 2.86 (s, 2H), 2.55 (s, 4H), 2.01 (s, 3H), 1.71 (s, 4H); Mass Spectrum: M−H⁺ 391

### j) 2,4-Dimethoxybenzyl {4-methyl-3-[1-oxo-7-(2-pyrrolidin-1-ylethoxy)isoquinolin-2(1H)-yl]phenyl}carbamate

To a suspension of the product of step i) (1.4 g) in anhydrous tetrahydrofuran (100 ml) was added triethylamine (17.9 ml) followed by diphenylphosphoryl azide (2.46 g). The mixture was stirred under nitrogen at room temperature for 48 hours. The solvent was removed in vacuo and the residue dissolved in toluene (100 ml). To the solution was added 2,4-dimethoxybenzyl alcohol (2.4 g) and the mixture heated at reflux under nitrogen for 90 minutes. After cooling to room temperature the reaction mixture was poured onto a SiO₂ chromotography column and the product eluted with 1% triethylamine and 2% methanol in dichloromethane to give 2,4-dimethoxybenzyl {4-methyl-3-[1-oxo-7-(2-pyrrolidin-1-ylethoxy)isoquinolin-2(1H)-yl]phenyl}carbamate as a gum (1.85 g). Mass Spectrum: M+H⁺ 558

### k) 2-(5-Amino-2-methylphenyl)-7-(2-pyrrolidin-1-ylethoxy)isoquinolin-1(2H)-one

A solution of the product of step j) (1.85 g) in dichloromethane (150 ml) was treated with trifluoroacetic acid (10 ml) and the mixture stirred at room temperature for 3 hours. The reaction mixture was concentrated to dryness in vacuo. The residue was then partitioned between dichloromethane and aqueous sodium bicarbonate. The organics were dried over magnesium sulphate, filtered and concentrated to dryness. The residue was then purified by SiO₂ chromotography eluting with 1% triethylamine and 2% methanol in dichloromethane followed by 1% triethylamine and 6% methanol in dichloromethane to elute 2-(5-amino-2-methylphenyl)-7-(2-pyrrolidin-1-ylethoxy)isoquinolin-1(2H)-one which was isolated as a gum that solidified on standing (1.0 g). NMR Spectrum: (CDCl₃) 7.88 (d, 1H), 7.49 (d, 1H), 7.33 (q, 1H), 7.11 (d, 1H), 6.91 (d, 1H), 6.69 (q, 1H), 6.58 (d, 1H), 6.51 (d, 1H), 4.25 (t, 2H), 3.66 (s, 2H), 2.96 (t, 2H), 2.67-2.63 (m, 4H), 2.03 (s, 3H), 1.84-1.80 (m, 4H); Mass Spectrum: M+H⁺ 364

### l) N-{4-Methyl-3-[1-oxo-7-(2-pyrrolidin-1-ylethoxy)isoquinolin-2(1H)-yl]phenyl}-3-piperidin-1-ylbenzamide

A mixture of the product of step k) (0.18 g) and 3-piperidin-1-ylbenzoic acid (0.10 g) in anhydrous 1-methyl-2-pyrrolidinone (7 ml) containing N,N-diisopropylethylamine (0.19 g) was treated with O-(7-azaenzotriazol-1-yl)-N,N,N′,N′-tetramethyluronium hexafluorophosphate (0.24 g) and the reaction mixture was stirred at room temperature for 72 hours. The mixture was then partitioned between ethyl acetate and brine. The organic layer was washed with brine, dried over magnesium sulphate, filtered and concentrated to dryness in vacuo. The product was purified by SiO₂ chromotography, eluting with 1% triethylamine and 4% methanol in dichloromethane to give N-{4-methyl-3-[1-oxo-7-(2-pyrrolidin-1-ylethoxy)isoquinolin-2(1H)-yl]phenyl}-3-piperidin-1-ylbenzamide as a colourless solid (0.06 g). NMR Spectrum: (DMSO-d₆) 7.79-7.76 (m, 2H), 7.72-7.67 (m, 2H), 7.43-7.30 (m, 5H), 7.21 (d, 1H), 7.14-7.12 (m, 1H), 6.71 (d, 1H), 4.20 (t, 2H), 3.21 (t, 4H), 2.85 (t, 2H), 2.55 (s, 4H), 2.03 (s, 3H), 1.69 (s, 4H), 1.65-1.60 (m, 4H), 1.58-1.52 (m, 2H); Mass Spectrum: M+H⁺ 551

### EXAMPLE 2

### N-{4-Methyl-3-[1-oxo-7-(2-pyrrolidin-1-ylethoxy)isoquinolin-2(1H)-yl]phenyl}-3-pyrrolidin-1-ylbenzamide hydrochloride

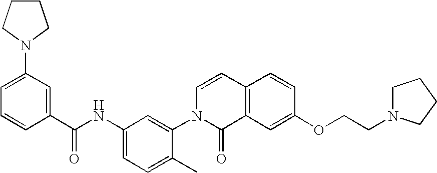

The title compound was prepared following the method of example 1 step 1), using the product of example 1 step k) (0.18 g) and 3-pyrrolidin-1-ylbenzoic acid (0.10 g). The product was purified by reverse phase HPLC, eluting with a gradient from 0.2% trifluoroacetic acid to acetonitrile and converted into its hydrochloride salt by dissolving in acetonitrile, adding 4M hydrogen chloride in dioxane and concentrating to dryness in vacuo to give N-{4-methyl-3-[1-oxo-7-(2-pyrrolidin-1-ylethoxy)isoquinolin-2(1H)-yl]phenyl}-3-pyrrolidin-1-ylbenzamide hydrochloride as a colourless solid (0.11 g). NMR Spectrum: (DMSO-d₆) 10.57 (s, 1H), 10.26 (s, 1H), 7.81-7.75 (m, 4H), 7.50 (dd, 1H), 7.38 (d, 1H), 7.34-7.25 (m, 2H), 7.17 (d, 1H), 7.06 (s, 1H), 6.74 (d, 2H), 4.51-4.48 (m, 2H), 3.65-3.60 (m, 4H), 3.30 (s, 4H), 3.17-3.13 (m, 2H), 2.03-1.96 (m, 9H), 1.92-1.89 (m, 2H); Mass Spectrum: M+H⁺ 537.

### EXAMPLE 3

### 3-Azepan-1-yl-N-{4-methyl-3-[1-oxo-7-(2-pyrrolidin-1-ylethoxy)isoquinolin-2(1H)-yl]phenyl}benzamide hydrochloride

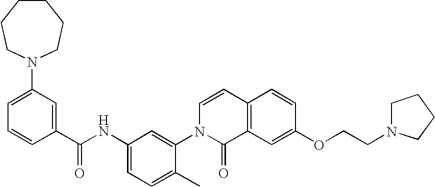

### a) Ethyl 3-azepan-1-ylbenzoate

A mixture of ethyl 3-bromobenzoate (3.9 g), racemic 2,2′-bis(diphenylphosphino)-1,1′-binaphthyl (0.32 g), palladium acetate (0.15 g), hexamethyleneimine (2.6 g) and cesium carbonate (8.9 g) in toluene (25 ml) was deoxygenated with nitrogen and then heated under a nitrogen atmosphere at reflux for 16 hours. The reaction mixture was filtered through diatomaceous earth (Celite®) washing with dichloromethane and the organics concentrated to dryness in vacuo. The residue was then purified by chromotography eluting with a 0 to 5% gradient of 7N methanolic ammonia in dichloromethane to give ethyl 3-azepan-1-ylbenzoate as a colourless oil (2.8 g). NMR Spectrum: (CDCl₃) 7.20-7.70 (m, 4H), 4.37 (dq, J=8.2, 7.2 Hz, 2H), 3.49 (t, J=5.9 Hz, 4H), 1.74-1.85 (m, 4H), 1.51-1.56 (m, 4H), 1.39 (td, J=7.1, 5.2 Hz, 3H); Mass Spectrum: M+H⁺ 248.

### b) 3-Azepan-1-ylbenzoic acid

To a solution of the product of step a) (2.79 g) in methanol (10 ml) was added a 5N aqueous solution of sodium hydroxide (5 ml) and then stirred at room temperature for 16 hours. The mixture was acidified with acetic acid and then concentrated to approximately half volume. The resulting precipitate was collected by filtration, washing with water to give 3-azepan-1-ylbenzoic acid as a colourless solid (1.64 g). NMR Spectrum: (DMSO-d₆) 7.20-7.26 (m, 2H), 7.13 (d, J=7.4 Hz, 1H), 6.90 (dd, J=8.6, 3.2 Hz, 1H), 3.46 (t, J=6.0 Hz, 4H), 1.73 (s, 4H), 1.43-1.50 (m, 4H); Mass Spectrum: M+H⁺ 220.

### c) 3-Azepan-1-yl-N-{4-methyl-3-[1-oxo-7-(2-pyrrolidin-1-ylethoxy)isoquinolin-2(1H)-yl]phenyl}benzamide hydrochloride

The title compound was prepared following the method of example 1 step 1), using the product of example 1 step k) (0.18 g) and the product of step b) (0.11 g). The product was purified by SiO₂ chromotography eluting with 1% ammonia and 4% methanol in dichloromethane and converted into its hydrochloride salt by dissolving in acetonitrile, adding 4M hydrogen chloride in dioxane and concentrating to dryness in vacuo to give 3-azepan-1-yl-N-{4-methyl-3-[1-oxo-7-(2-pyrrolidin-1-ylethoxy)isoquinolin-2(1H)-yl]phenyl}benzamide hydrochloride as a colourless solid (0.05 g). NMR Spectrum: (DMSO-d₆) 10.62 (s, 1H), 10.26 (s, 1H), 7.80-7.74 (m, 4H), 7.50 (dd, 1H), 7.38 (d, 1H), 7.30-7.24 (m, 2H), 7.19-7.14 (m, 2H), 6.91 (d, 1H), 6.74 (d, 1H), 4.51-4.48 (m, 2H), 3.64-3.60 (m, 4H), 3.39 (t, 4H), 3.16-3.12 (m, 2H), 2.03 (s, 5H), 1.92-1.87 (m, 2H), 1.75 (s, 4H), 1.48 (s, 4H); Mass Spectrum: M+H⁺ 565.

### EXAMPLE 4

### 3-[(3R)-3-Hydroxypyrrolidin-1-yl]-N-{4-methyl-3-[1-oxo-7-(2-pyrrolidin-1-ylethoxy)isoquinolin-2(1H)-yl]phenyl}benzamide hydrochloride

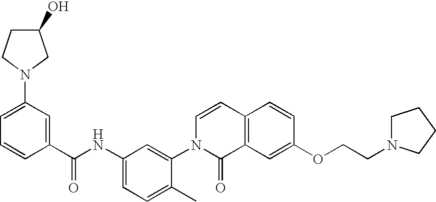

### a) Ethyl 3-[(3R)-3-hydroxypyrrolidin-1-yl]benzoate

The sub-title compound was prepared by the method of example 3 step a) using (R)-3-hydroxypyrollidine (3.4 g) to give ethyl 3-[(3R)-3-hydroxypyrrolidin-1-yl]benzoate as a colourless solid (4.0 g). NMR Spectrum: ¹HNMR (CDCl₃) 7.17-7.40 (m, 3H), 6.71-6.78 (m, 1H), 5.67 (s, 1H), 4.62 (s, 1H), 4.37 (q, J=7.1 Hz, 2H), 3.26-3.59 (m, 4H), 2.03-2.36 (m, 2H), 1.39 (t, J=7.1 Hz, 3H); Mass Spectrum: M+H⁺ 236.

### b) 3-[(3R)-3-Hydroxypyrrolidin-1-yl]benzoic acid

The sub-title compound was prepared by the method of example 3 step b) using the product of step a) (1.6 g) to give 3-[(3R)-3-hydroxypyrrolidin-1-yl]benzoic acid as a colourless solid (1.2 g). NMR Spectrum: (DMSO-d₆) 7.26 (t, J=7.8 Hz, 1H), 7.16 (dt, J=7.6, 1.2 Hz, 1H), 7.04 (t, J=2.0 Hz, 1H), 6.73 (ddd, J=8.1, 2.6, 1.0 Hz, 1H), 4.41 (septet, J=2.5 Hz, 2H), 3.43 (dd, J 10.1, 4.9 Hz, 2H), 3.24-3.36 (m, 1H), 3.08 (dd, J=10.1, 1.4 Hz, 1H), 1.99-2.11 (m, 1H), 1.86-1.95 (m, 1H); Mass Spectrum: M+H⁺ 208.

### c) 3-[(3R)-3-Hydroxypyrrolidin-1-yl]-N-{4-methyl-3-[1-oxo-7-(2-pyrrolidin-1-ylethoxy)isoquinolin-2(1H)-yl]phenyl}benzamide hydrochloride

The title compound was prepared following the method of example 1 step 1), using the product of example 1 step k) (0.18 g) and the product of step b) (0.10 g). The product was purified by reverse phase HPLC, eluting with a gradient from 0.2% TFA to acetonitrile and converted into its hydrochloride salt by dissolving in acetonitrile, adding 4M HCl in dioxane and concentrating to dryness in vacuo to give 3-[(3R)-3-hydroxypyrrolidin-1-yl]-N-{4-methyl-3-[1-oxo-7-(2-pyrrolidin-1-ylethoxy)isoquinolin-2(1H)-yl]phenyl}benzamide hydrochloride as a colourless solid (0.1 μg). NMR Spectrum: (DMSO-d₆) 10.53 (s, 1H), 10.26 (s, 1H), 7.81-7.75 (m, 4H), 7.50 (dd, 1H), 7.38 (d, 1H), 7.34-7.25 (m, 2H), 7.16 (d, 1H), 7.02 (s, 1H), 6.75-6.70 (m, 2H), 4.51-4.47 (m, 2H), 4.43-4.41 (m, 1H), 3.65-3.60 (m, 4H), 3.48-3.44 (m, 1H), 3.41-3.31 (m, 2H), 3.16-3.12 (m, 2H), 2.08-2.00 (m, 7H), 1.92-1.87 (m, 4H); Mass Spectrum: M+H⁺ 553.

### EXAMPLE 5

### N-{4-Methyl-3-[1-oxo-7-(2-pyrrolidin-1-ylethoxy)isoquinolin-2(1H)-yl]phenyl}-2-pyrrolidin-1-ylisonicotinamide

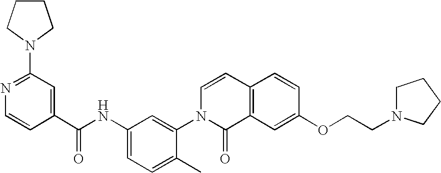

### a) 2-Chloro-N-{4-methyl-3-[1-oxo-7-(2-pyrrolidin-1-ylethoxy)isoquinolin-2(1H)-yl]phenyl}isonicotinamide

To a suspension of 2-chloro-isonicotinic acid (0.08 g) in dichloromethane (25 ml) containing 1 drop of N,N-dimethylformamide was added a 2M solution of oxalyl chloride in dichloromethane (1 ml) and the mixture stiffed at room temperature for 1 hour. After concentrating to dryness in vacuo and azeotroping x3 with dichloromethane, the residue was dissolved in dichloromethane (20 ml) and added slowly to a solution of the product of example 1 step 1) (0.11 g) in N,N-diisopropylethylamine (0.5 ml) and stirred at room temperature for 16 hours. The reaction mixture was concentrated to dryness, the residue was then taken up in methanol and purified by reverse phase HPLC, eluting with a 0.1% ammonia in acetonitrile gradient to give 2-chloro-N-{4-methyl-3-[1-oxo-7-(2-pyrrolidin-1-ylethoxy)isoquinolin-2(1H)-yl]phenyl}isonicotinamide as a colourless solid (0.10 g). NMR Spectrum: (DMSO-d₆) 10.65 (s, 1H), 8.62 (d, J=5.2 Hz, 1H), 8.00 (s, 1H), 7.87 (dd, J 5.1, 1.4 Hz, 1H), 7.66-7.77 (m, 4H), 7.39-7.45 (m, 2H), 7.21 (d, J=7.5 Hz, 1H), 6.72 (d, J=7.3 Hz, 1H), 4.19 (t, J=5.7 Hz, 2H), 2.83 (t, J=5.8 Hz, 2H), 2.47-2.55 (m, 4H), 2.04 (s, 3H), 1.65-1.73 (m, 4H); Mass Spectrum: M+H⁺ 503.

### b) N-{4-Methyl-3-[1-oxo-7-(2-pyrrolidin-1-ylethoxy)isoquinolin-2(1H)-yl]phenyl}-2-pyrrolidin-1-ylisonicotinamide

To a suspension of the product of step a) (0.08 g) in acetonitrile (3 ml) was added pyrrolidine (0.2 ml) and the mixture heated under microwave irradiation at 120° C. for 4.5 hours. The resulting precipitate was collected by filtration and washed with acetonitrile to give N-{4-methyl-3-[1-oxo-7-(2-pyrrolidin-1-ylethoxy)isoquinolin-2(1H)-yl]phenyl}-2-pyrrolidin-1-ylisonicotinamide as a colourless solid (0.08 g). NMR Spectrum: (DMSO-d₆) 10.39 (s, 1H), 8.20 (d, J=5.1 Hz, 1H), 7.78 (dd, J=8.5, 2.1 Hz, 1H), 7.74 (d, J=1.8 Hz, 1H), 7.71 (d, J=8.7 Hz, 1H), 7.67 (d, J=2.6 Hz, 1H), 7.38-7.44 (m, 2H), 7.21 (d, J=7.4 Hz, 1H), 6.96 (dd, J=5.1, 1.0 Hz, 1H), 6.86 (s, 1H), 6.71 (d, J=7.4 Hz, 1H), 4.19 (t, J=5.5 Hz, 2H), 3.44 (t, J=6.5 Hz, 4H), 2.83 (t, J=5.8 Hz, 2H), 2.52-2.56 (m, 4H), 2.03 (s, 3H), 1.93-1.98 (m, 4H), 1.66-1.71 (m, 4H); Mass Spectrum: M+H⁺ 538

### EXAMPLE 6

### N-{4-Methyl-3-[1-oxo-7-(2-pyrrolidin-1-ylethoxy)isoquinolin-2(1H)-yl]phenyl}-3-(tetrahydro-2H-pyran-4-yl)benzamide

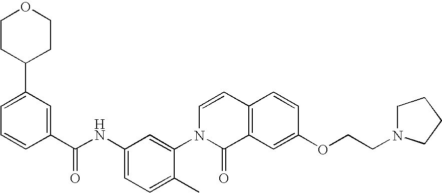

### a) 3-(Tetrahydro-2H-pyran-4-yl)benzoic acid

To a stirred solution of 3,6-dihydro-2H-pyran-4-yl trifluoromethanesulfonate (10.7 g) (prepared according to WO9709328) and tetrakis(triphenylphosphine)palladium (1.78 g) in toluene (594 ml) was added 3-cynophenylboronic acid (7.42 g), dissolved a minimum amount of ethanol, followed by a 2M aqueous sodium carbonate solution (50.5 ml). The resulting mixture was heated at reflux for 16 hours. After filtration, the mixture was concentrated to dryness in vacuo and the residue partitioned between dichloromethane and brine. The organics were dried over magnesium sulphate, filtered and concentrated to dryness. The residue was then purified by SiO₂ chromotograhy, eluting with a iso-hexane to 15% ethyl acetate in iso-hexane gradient to yield 3-(3,6-dihydro-2H-pyran-4-yl)benzonitrile (5.91 g). A solution of 3-(3,6-dihydro-2H-pyran-4-yl)benzonitrile (1.64 g) in n-butanol (5 ml) and 5M aqueous sodium hydroxide solution (5 ml) was heated at reflux for 16 hours. The cooled reaction mixture was concentrated to dryness in vacuo and the residue taken up into water and acidified using a 2M aqueous hydrochloric acid solution. The mixture was then extracted into ethyl acetate (2×100 ml) and the combined organics dried over magnesium sulphate, filtered and concentrated to dryness to give 3-(3,6-dihydro-2H-pyran-4-yl)benzoic acid (1.60 g). To a solution of 3-(3,6-dihydro-2H-pyran-4-yl)benzoic acid (6.31 g) in ethanol (200 ml) was added 10% palladium on carbon (0.40 g) and the mixture stirred under a balloon of hydrogen at room temperature for 72 hours. The reaction mixture was filtered through diatomaceous earth (Celite®) and the filtrate concentrated to dryness in vacuo to afford 3-(tetrahydro-2H-pyran-4-yl)benzoic acid (6.12 g). NMR Spectum: (DMSO-d₆) 12.90 (s, 1H), 7.83-7.76 (m, 2H), 7.53 (d, J=7.3 Hz, 1H), 7.44 (t, J=7.6 Hz, 1H), 3.99-3.91 (m, 2H), 3.44 (td, J=11.2, 3.1 Hz, 2H), 2.91-2.79 (m, 1H), 1.77-1.59 (m, 4H)

### b) N-{4-Methyl-3-[1-oxo-7-(2-pyrrolidin-1-ylethoxy)isoquinolin-2(1H)-yl]phenyl}-3-(tetrahydro-2H-pyran-4-yl)benzamide

The title compound was prepared following the method of example 5 step a), using the product of example 1 step k) (0.11 g) and the product of step a) (0.10 g). The reaction mixture was concentrated to dryness, the residue was then taken up in methanol and purified by reverse phase HPLC, eluting with a 0.1% ammonia to acetonitrile gradient to give N-{4-methyl-3-[1-oxo-7-(2-pyrrolidin-1-ylethoxy)isoquinolin-2(1H)-yl]phenyl}-3-(tetrahydro-2H-pyran-4-yl)benzamide as a colourless solid (0.08 g). NMR Spectrum: (DMSO-d₆) 10.32 (s, 1H), 7.84 (s, 1H), 7.80 (q, J=2.2 Hz, 1H), 7.76-7.79 (m, 2H), 7.71 (d, J=8.8 Hz, 1H), 7.68 (d, J=2.7 Hz, 1H), 7.47 (d, J=4.8 Hz, 1H), 7.47-7.51 (m, 1H), 7.41 (dd, J=8.7, 2.9 Hz, 1H), 7.39 (d, J=7.8 Hz, 1H), 7.22 (d, J=7.3 Hz, 1H), 6.72 (d, J=7.3 Hz, 1H), 4.19 (t, J=5.9 Hz, 2H), 3.97 (dd, J=8.2, 2.8 Hz, 2H), 3.39-3.52 (m, 2H), 2.84 (t, J=5.8 Hz, 2H), 2.47-2.57 (m, 5H), 2.03 (s, 3H), 1.66-1.78 (m, 8H); Mass Spectrum: M+H⁺ 552.

### EXAMPLE 7

### N-{4-methyl-3-[1-oxo-7-(3-pyrrolidin-1-ylpropoxy)isoquinolin-2(1H)-yl]phenyl}-3-(2-thienyl)benzamide

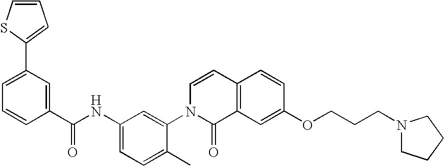

### a) 3-Bromo-N-(4-methyl-3-nitrophenyl)benzamide

To a stirred suspension of 3-bromobenzoic acid (16.7 g) in dichloromethane (200 ml) containing DMF (1 drop) was added oxalyl chloride (8 ml). After 2 hours the mixture was concentrated to dryness. The residue was redissolved in dichloromethane (200 ml) and treated with 4-methyl-3-nitroaniline (12.7 g) and N,N-diisopropylethylamine (30 ml). After stirring at ambient temperature overnight the reaction was concentrated to dryness and purified by SiO₂ chromatography, eluting with dichloromethane to give 3-bromo-N-(4-methyl-3-nitrophenyl)benzamide as a solid (20.5 g); Mass Spectrum: M+H⁺ 334/336.

### b) N-(3-Amino-4-methylphenyl)-3-bromobenzamide

A mixture of the product of step a) (20 g), reduced iron powder (20 g) and ammonium chloride (20 g) in ethanol (200 ml) and water (200 ml) was heated at reflux for 1 hour. The reaction mixture was passed through a plug of silica washing with ethanol and the liquors concentrated to dryness to give N-(3-amino-4-methylphenyl)-3-bromobenzamide as a solid (21 g); Mass Spectrum: M+H⁺ 304/306

### c) 3-Bromo-N-[3-(7-methoxy-1,3-dioxo-3,4-dihydroisoquinolin-2(1H)-yl)-4-methylphenyl]benzamide

The sub-title compound was prepared following the procedure of example 1 step d) using the product of step b) (1.0 g) and the product of example 1 step c) (0.63 g) to give 3-bromo-N-[3-(7-methoxy-1,3-dioxo-3,4-dihydroisoquinolin-2(1H)-yl)-4-methylphenyl]benzamide as a solid (1.0 g); Mass Spectrum: M+H⁺ 478/480

### d) 3-Bromo-N-[3-(7-methoxy-1-oxoisoquinolin-2(1H)-yl)-4-methylphenyl]benzamide

The sub-title compound was prepared following the procedure of example 1 step e) using the product of step c) (11.0 g) to give 3-bromo-N-[3-(7-methoxy-1-oxoisoquinolin-2(1H)-yl)-4-methylphenyl]benzamide as a solid (0.91 g); NMR Spectrum: (DMSO-d₆) 10.47 (s, 1H), 8.15 (t, 1H), 7.95 (dt, 1H), 7.82-7.75 (m, 3H), 7.72 (d, 1H), 7.68 (d, 1H), 7.51 (t, 1H), 7.42 (d, 1H), 7.40 (dd, 1H), 7.22 (d, 1H), 6.72 (d, 1H), 3.89 (s, 3H), 2.04 (s, 3H); Mass Spectrum: M+H⁺ 464/466

### e) N-[3-(7-Methoxy-1-oxoisoquinolin-2(1H)-yl)-4-methylphenyl]-3-(2-thienyl)benzamide

A mixture of the product of step d) (0.50 g), potassium carbonate (0.30 g), thiophene-2-boronic acid (0.18 g) and tetrakis(triphenylphosphine)palladium(0) (0.06 g) in tetrahydrofuran (4 ml) and water (2 ml) was heated under microwave irradiation at 120° C. for 30 minutes. The mixture was then passed through a silica plug, washing with 5% methanol in dichloromethane. The product was purified by HPLC, eluting with a 0.1% ammonia in acetonitrile gradient to give N-[3-(7-methoxy-1-oxoisoquinolin-2(1H)-yl)-4-methylphenyl]-3-(2-thienyl)benzamide as a solid (0.35 g); NMR Spectrum: (DMSO-d₆) 10.48 (s, 1H), 8.19 (s, 1H), 7.90-7.85 (m, 2H), 7.83-7.77 (m, 2H), 7.72 (d, 1H), 7.68 (d, 1H), 7.65-7.55 (m, 3H), 7.44-7.38 (m 2H), 7.23 (d, 1H), 7.19 (dd, 1H), 6.73 (d, 1H), 3.89 (s, 3H), 2.04 (s, 3H); Mass Spectrum: M+H⁺ 467.

### f) N-[3-(7-Hydroxy-1-oxoisoquinolin-2(1H)-yl)-4-methylphenyl]-3-(2-thienyl)benzamide

The sub-title compound was prepared following the procedure of example 1 step f) using the product of step e) (0.33 g) to give N-[3-(7-hydroxy-1-oxoisoquinolin-2(1H)-yl)-4-methylphenyl]-3-(2-thienyl)benzamide as a solid (0.31 g); Mass Spectrum: M+H⁺ 453

### g) N-{4-Methyl-3-[1-oxo-7-(3-pyrrolidin-1-ylpropoxy)isoquinolin-2(1H)-yl]phenyl}-3-(2-thienyl)benzamide

A mixture of the product of step f) (0.16 g), potassium carbonate (0.48 g) and 1-bromo-3-chloropropane (0.28 g) in acetonitrile (5 ml) was heated at reflux for 1 hour. Pyrrolidine (0.35 ml) was added and the mixture heated at 100° C. under microwave irradiation for 2 hours. The reaction mixture was filtered and purified by HPLC, eluting with a 0.1% ammonia in acetonitrile gradient to give N-{4-methyl-3-[1-oxo-7-(3-pyrrolidin-1-ylpropoxy)isoquinolin-2(1H)-yl]phenyl}-3-(2-thienyl)benzamide as a solid (0.062 g); NMR Spectrum: (DMSO-d₆) 10.48 (s, 1H), 8.19 (s, 1H), 7.91-7.85 (m, 2H), 7.82-7.77 (m, 2H), 7.71 (d, 1H), 7.68-7.55 (m, 4H), 7.43-7.38 (m, 2H), 7.25-7.17 (m, 2H), 6.72 (d, 1H), 4.15 (t, 2H), 2.59-2.40 (m, 6H), 2.04 (s, 3H), 1.94 (quintet, 2H), 1.68 (s, 4H); Mass Spectrum: M+H⁺ 564.

### EXAMPLE 8

### N-{3-[7-[3-(dimethylamino)propoxy]-1-oxoisoquinolin-2(1H)-yl]-4-methylphenyl}-3-(2-thienyl)benzamide

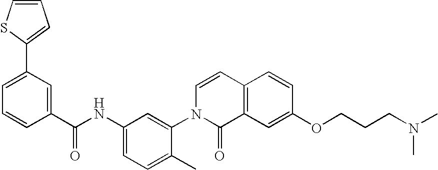

The title compound was prepared following the procedure of example 7 step g) using the product of example 7 step f) (0.16 g) and a 33% w/w solution of dimethylamine in ethanol (1 ml) to give N-{3-[7-[3-(dimethylamino)propoxy]-1-oxoisoquinolin-2(1H)-yl]-4-methylphenyl}-3-(2-thienyl)benzamide as a solid (0.062 g); NMR Spectrum: (DMSO-d₆) 10.48 (s, 1H), 8.19 (s, 1H), 7.91-7.85 (m, 2H), 7.82-7.77 (m, 2H), 7.71 (d, 1H), 7.68-7.55 (m, 4H), 7.43-7.38 (m, 2H), 7.24-7.17 (m, 2H), 6.72 (d, 1H), 4.13 (t, 2H), 2.41 (t, 2H), 2.17 (s, 6H), 2.08-2.02 (m, 2H), 1.91 (s, 3H); Mass Spectrum: M+H⁺ 538

### EXAMPLE 9

### N-{3-[7-[2-(dimethylamino)ethoxy]-1-oxoisoquinolin-2(1H)-yl]-4-methylphenyl}-2-(2-thienyl)isonicotinamide

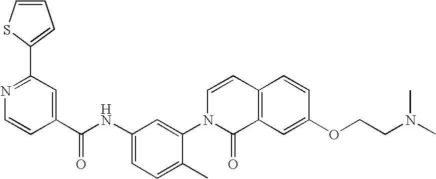

### a) N-(3-Amino-4-methylphenyl)-2-(2-thienyl)isonicotinamide

Tetrakis(triphenylphosphine)palladium(0) (0.25 g) was added to a mixture of N-(4-methyl-3-nitrophenyl)-2-(2-thienyl)isonicotinamide (4.47 g), thiophene-2-boronic acid (2.86 g), anhydrous sodium carbonate (5.35 g) and anhydrous ethanol (50 ml) and the mixture stirred at reflux for 2 hours. After cooling, the mixture was quenched with water (150 ml) and extracted into dichloromethane. The organic phase was dried over Na₂SO₄, filtered and concentrated in vacuo. The product was purified by SiO₂ chromatography eluting with 0 to 10% methanol in dichloromethane to yield N-(3-amino-4-methylphenyl)-2-(2-thienyl)isonicotinamide as a solid (3.0 g); NMR Spectrum: (DMSO-d₆) 10.20 (s, 1H), 8.67 (dd, 1H), 8.30 (s, 1H), 7.92 (dd, 1H), 7.69 (dd, 1H), 7.67 (dd, 1H), 7.22 (dd, 1H), 7.11 (d, 1H), 6.90 (d, 1H), 6.84 (dd, 1H), 4.91 (s, 2H), 2.04 (s, 3H)

### b) N-[3-(7-Methoxy-1-oxoisoquinolin-2(1)-yl)-4-methylphenyl]-2-(2-thienyl)isonicotinamide

A mixture of the product of example 1 step c) (0.38 g) and the product of step a) (0.55 g) in toluene (12 ml) and acetic acid (3 ml) was stirred at reflux for 30 hours and then concentrated to dryness in vacuo. The solid residue was treated with dichloromethane (20 ml) and methanol (5 ml) and then solid sodium borohydride (4×100 mg) was added batchwise. The mixture was stirred at room temperature for 30 minutes and then concentrated hydrochloric acid (1.1 ml) was added. The mixture was stirred for 17 hours and then poured onto saturated aqueous sodium bicarbonate solution and extracted with dichloromethane. The organic phase was dried over Na₂SO₄, filtered and concentrated in vacuo. The product was purified by SiO₂ chromatography eluting with 0 to 100% ethyl acetate in i-hexane to give N-[3-(7-methoxy-1-oxoisoquinolin-2(1H)-yl)-4-methylphenyl]-2-(2-thienyl)isonicotinamide (0.49 g); NMR Spectrum: (DMSO-d₆) 10.64 (s, 1H), 8.71 (d, 1H), 8.34 (s, 1H), 7.92 (dd, 1H), 7.83-7.66 (m, 6H), 7.46-7.38 (m, 2H), 7.27-7.18 (m, 2H), 6.77-6.69 (m, 1H), 3.88 (s, 3H), 2.05 (s, 3H)

### c) N-{3-[7-(2-Bromoethoxy)-1-oxoisoquinolin-2(1H)-yl]-4-methylphenyl}-2-(2-thienyl)isonicotinamide

A mixture of the product of step b) (1.0 g), sodium ethanethiolate (0.82 g) and DMF (4 ml) was heated under microwave irradiation for 20 minutes at 140° C. before being cooled to room temperature. The mixture was treated with 2M HCl (10 ml), methanol (5 ml) and ethyl acetate (80 ml), then washed with saturated aqueous sodium bicarbonate solution and then water. The organic phase was dried over Na₂SO₄, filtered and concentrated in vacuo. The residue was treated with DMF (10 ml), potassium carbonate (2.0 g) and 1,2-dibromoethane (1.0 ml) and the resulting mixture stirred at 75° C. for 2 hours. After being cooled to room temperature the mixture was diluted with dichloromethane and washed with water. The organic phase was dried over MgSO₄, filtered and concentrated to dryness.

The product was purified by SiO₂ chromatography eluting first with 0-100% ethyl acetate in i-hexane and then 10% methanol in dichloromethane to give N-{3-[7-(2-bromoethoxy)-1-oxoisoquinolin-2(1H)-yl]-4-methylphenyl}-2-(2-thienyl)isonicotinamide as a colourless solid (0.70 g); NMR Spectrum: (DMSO-d₆) 10.67 (s, 1H), 8.71 (d, 1H), 8.34 (s, 1H), 7.98-7.88 (m, 1H), 7.84-7.17 (m, 10H), 6.74 (d, 1H), 3.93-3.77 (m, 2H), 2.05 (s, 3H), 4.47 (t, 211)

### d) N-{3-[7-[2-(Dimethylamino)ethoxy]-1-oxoisoquinolin-2(1H)-yl]-4-methylphenyl}-2-(2-thienyl)isonicotinamide

A mixture of the product of step c) (0.40 g), dimethylamine hydrochloride (0.38 g), triethylamine (1 ml) and DMF (1 ml) was stirred at 75° C. for 2 hours. The reaction mixture was then taken up in methanol and purified by HPLC, eluting with a 0.1% ammonia to acetonitrile gradient to give N-{3-[7-[2-(dimethylamino)ethoxy]-1-oxoisoquinolin-2(1H)-yl]-4-methylphenyl}-2-(2-thienyl)isonicotinamide as a solid (0.10 g). NMR Spectrum: (DMSO-d₆) 10.66 (s, 1H), 8.71 (d, 1H), 8.33 (s, 1H), 7.92 (dd, 1H), 7.80 (t, 1H), 7.74-7.66 (m, 5H), 7.44 (d, 1H), 7.41 (dd, 1H), 7.25-7.19 (m, 2H), 6.73 (d, 1H), 4.17 (m, 2H), 2.68 (t, 2H), 2.24 (s, 6H), 2.05 (s, 3H); Mass Spectrum: M+H⁺ 525

### EXAMPLE 10

### N-{4-Methyl-3-[1-oxo-7-(2-pyrrolidin-1-ylethoxy)isoquinolin-2(1H)-yl]phenyl}-2-(2-thienyl)isonicotinamide

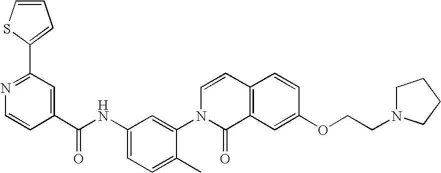

The title compound was prepared following the procedure of example 9 step d) using the product of example 9 step c) (0.40 g) and pyrrolidine (1 ml) to yield N-{4-methyl-3-[1-oxo-7-(2-pyrrolidin-1-ylethoxy)isoquinolin-2(1H)-yl]phenyl}-2-(2-thienyl)isonicotinamide as a solid (0.05 g); NMR Spectrum: (DMSO-d₆) 10.65 (s, 1H), 8.71 (d, 1H), 8.33 (s, 1H), 7.92 (d, 1H), 7.83-7.64 (m, 6H), 7.47-7:38 (m, 2H), 7.25-7.18 (m, 2H), 6.73 (d, 1H), 4.20 (t, 2H), 2.84 (t, 2H), 2.51 (m, 4H), 2.05 (s, 3H), 1.69 (m, 4H); Mass Spectrum: M+H⁺ 551

### EXAMPLE 11

### N-{4-Methyl-3-[1-oxo-7-(3-pyrrolidin-1-ylpropoxy)isoquinolin-2(1H)-yl]phenyl}-3-(1,3-oxazol-5-yl)benzamide

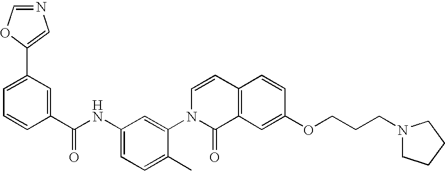

### a) N-(4-Methyl-3-nitrophenyl)-3-(1,3-oxazol-5-yl)benzamide

The sub-title compound was prepared following the method of example 7 step a) using 3-(1,3-oxazol-5-yl)benzoic acid (5.0 g). The resulting precipitate was filtered, washed with water and dichloromethane and dried to yield N-(4-methyl-3-nitrophenyl)-3-(1,3-oxazol-5-yl)benzamide as a solid (6.1 g); Mass Spectrum: M+H⁺ 324

### b) N-(3-Amino-4-methylphenyl)-3-(1,3-oxazol-5-yl)benzamide

The sub-title compound was prepared following the method of example 7 step b) using the product of step a) (6.1 g). Recrystallisation from dichloromethane gave N-(3-amino-4-methylphenyl)-3-(1,3-oxazol-5-yl)benzamide as a solid (3.5 g); Mass Spectrum: M+H⁺ 294

### c) N-[3-(7-Methoxy-1,3-dioxo-3,4-dihydroisoquinolin-2(1H)-yl)-4-methylphenyl]-3-(1,3-oxazol-5-yl)benzamide

The sub-title compound was prepared following the method of example 1 step d) using the product of step b) (2.9 g) and the product of example 1 step c) (1.9 g). Purification by SiO₂ chromatography eluting with ethyl acetate:dichloromethane (3:7) gave N-[3-(7-methoxy-1,3-dioxo-3,4-dihydroisoquinolin-2(1H)-yl)-4-methylphenyl]-3-(1,3-oxazol-5-yl)benzamide as a solid (2.4 g); Mass Spectrum: M+H⁺ 468

### d) N-[3-(7-Methoxy-1-oxoisoquinolin-2(1H)-yl)-4-methylphenyl]-3-(1,3-oxazol-5-yl)benzamide

The sub-title compound was prepared following the method of example 1 step e) using the product of step c) (2.3 g) to yield N-[3-(7-methoxy-1-oxoisoquinolin-2(1H)-yl)-4-methylphenyl]-3-(1,3-oxazol-5-yl)benzamide as a solid (0.95 g); NMR Spectrum: (DMSO-d₆) 10.51 (1H, s), 8.52 (1H, s), 8.28 (1H, t), 7.96 (1H, t), 7.94 (1H, t), 7.82-7.78 (3H, m), 7.74-7.62 (3H, m), 7.43-7.39 (2H, m), 7.24-7.21 (1H, m), 6.73 (1H, d), 3.89 (3H, s), 2.04 (3H, s) Mass Spectrum: M+H⁺ 452

### e) N-[3-(7-Methoxy-1-oxoisoquinolin-2(1H)-yl)-4-methylphenyl]-3-(1,3-oxazol-5-yl)benzamide

The sub-title compound was prepared following the method of example 1 step f) using the product of step d) (0.87 g) to afford N-[3-(7-methoxy-1-oxoisoquinolin-2(1H)-yl)-4-methylphenyl]-3-(1,3-oxazol-5-yl)benzamide (0.41 g); Mass Spectrum: M+H⁺ 438.

### f) N-{4-Methyl-3-[1-oxo-7-(3-pyrrolidin-1-ylpropoxy)isoquinolin-2(1H)-yl]phenyl}-3-(1,3-oxazol-5-yl)benzamide

The title compound was prepared following the method of example 7 step g) using the product of step e) (0.20 g). The product was purified by reverse phase HPLC to yield N-{4-methyl-3-[1-oxo-7-(3-pyrrolidin-1-ylpropoxy)isoquinolin-2(1H)-yl]phenyl}-3-(1,3-oxazol-5-yl)benzamide as a solid (0.04 g); NMR Spectrum: (DMSO-d₆) 10.51 (1H, s), 8.52 (1H, s), 8.28 (1H, t), 7.95 (1H, t), 7.94 (1H, t), 7.82-7.77 (3H, m), 7.71 (1H, d), 7.68-7.63 (2H, m), 7.42-7.38 (2H, m), 7.22 (1H, d), 6.72 (1H, d), 4.15 (2H, t), 2.56 (2H, t), 2.46-2.42 (4H, m), 2.04 (3H, s), 1.94 (2H, quintet), 1.71-1.65 (4H, m). Mass Spectrum: M+H⁺ 549

### P38 Alpha Enzyme Assay

Enzyme assays were performed in polypropylene 96 well plates. The following solutions were added to each well; 10 μL of compound dilutions in assay buffer (20 mM HEPES pH 7.4, containing 20 mM magnesium acetate, 0.005% (w/v) Tween-20, 10 mM DTT) containing 1% (v/v) DMSO or assay buffer containing 1% (v/v) DMSO alone, 70 μL of assay buffer containing 36 nM substrate (biotinylated-ATF2) and 10 μL of an appropriate dilution of human active recombinant p38α-6His tagged. Depending on batch of p38, an appropriate dilution was typically a 5 nM solution to give a final enzyme concentration of 0.5 nM. At this stage, background control wells also received 50 μL of AlphaScreen quench buffer (10 mM HEPES pH 7.4 containing 100 mM EDTA, 0.2% (w/v) bovine serum albumin). The plate was covered, pre-incubated for 4 hours at 37° C. and the enzyme reaction initiated by addition of 10 μL 1 mM ATP. After incubation for a further 45 minutes at 37° C., the reaction was stopped by addition of 50 μL quench reagent and 50 μL of the quenched reaction mixture transferred to an opaque, white 96-well plate. Detection reagent, 25 μL of 10 mM HEPES pH 7.4 containing 100 mM EDTA, 0.2% (w/v) bovine serum albumin, 0.3 nM anti phosphoATF2 antibody and 25 μg/mL of AlphaScreen protein A acceptor and donor beads, was added to all wells in a darkened room, the plate sealed and left in the dark for between 5 and 24 hours before AlphaScreen readings were taken using a Perkin Elmer EnVision reader. The compounds of the examples give over 50% inhibition of p38α and/or p38β at concentrations less than 1 μM. For example, the following table shows a pIC50 figure for a representative compound:

| Compound of | |
|---|---|
| Example No. | pIC₅₀ |
| 1 | 8.0 |
| 5 | 8.7 |
| 9 | 9.7 |
| 11 | 9.1 |

1: A compound of formula (I) 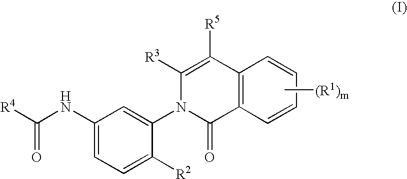
wherein
m is 0, 1 or 2;
R¹ is halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N,N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphonyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, aryl, aryl-(1-6C)alkyl, aryloxy, arylthio, arylsulphinyl, arylsulphonyl, aryl-(1-6C)alkoxy, arylamino, and wherein any aryl, heteroaryl or heterocyclyl group in a R¹ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl,
and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R¹ substituent may optionally bear 1 or 2 oxo or thioxo substituents;
R² is halogeno or (1-6C)alkyl;
R³ is hydrogen, halogeno, trifluoromethyl, cyano, (1-6C)alkyl, (1-6C)alkoxy, (1-6C)alkylthio, (1-6C)alkylamino, di[(1-6C)alkyl]amino, di-[(1-6C)alkyl]amino-(2-6C)alkoxy, (1-6C)alkylamino-(2-6C)alkoxy, amino-(2-6C)alkoxy, (1-6C)alkoxy-(2-6C)alkoxy, amino-(1-6C)alkyl, di[(1-6C)alkyl]amino-(1-6C)alkyl or (1-6C)alkylamino-(1-6C)alkyl;
R⁵ is hydrogen, halogeno, trifluoromethyl, cyano, (1-6C)alkyl, (1-6C)alkoxy, (1-6C)alkylthio, (1-6C)alkylamino, di[(1-6C)alkyl]amino, di-[(1-6C)alkyl]amino-(2-6C)alkoxy, (1-6C)alkylamino-(2-6C)alkoxy, amino-(2-6C)alkoxy, (1-6C)alkoxy-(2-6C)alkoxy, amino-(1-6C)alkyl, di[(1-6C)alkyl]amino-(1-6C)alkyl or (1-6C)alkylamino-(1-6C)alkyl;
R⁴ is aryl or heteroaryl, which aryl or heteroaryl is optionally substituted by one or more substituents independently selected from halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, hydroxy(1-6C)alkylamino-(1-6C)alkyl, carbamoyl-(1-6C)alkyl, N-(1-6C)alkylcarbamoyl-(1-6C)alkyl, N N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N,N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphinoyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, heterocyclylthio, aryl, aryl-(1-6C)alkyl, aryloxy, arylthio, arylthio, arylsulphinyl, aryl-(1-6C)alkoxy, arylamino, (3-6C)cycloalkyl, (3-6C)cycloalkyl-(1-6C)alkyl, (3-6C)cycloalkyloxy, (3-6C)cycloalkylthio, (3-6C)cycloalkyl-(1-6C)alkoxy and (3-6C)cycloalkylamino,
and wherein any heteroaryl, heterocyclyl, aryl or (3-6C)cycloalkyl group in a R⁴ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, (1-6C)alkylthio, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, (2-6C)alkanoyloxy, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl,
and wherein any of the R⁴ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy,
and wherein any heterocyclyl group in a R⁴ substituent may optionally bear 1 or 2 oxo or thioxo substituents;
or a pharmaceutically-acceptable salt thereof.

2. A compound of formula (I) according to claim 1 wherein
m is 1 or 2; and
R¹ is heterocyclyl, heterocyclyloxy, heterocyclyl-(1-6C)alkoxy, (1-6C)alkylamino-(2-6C)alkoxy, di-[(1-6C)alkyl]amino-(2-6C)alkoxy, (1-6C)alkylamino-(2-6C)alkyl or di[(1-6C)alkyl]amino-(1-6C)alkyl,
and wherein any heterocyclyl group in a R¹ substituent may optionally bear 1 or 2 substituents selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl,
and wherein any of the R¹ substituents defined hereinbefore which comprises a CH₂ group which is attached to 2 carbon atoms or a CH₃ group which is attached to a carbon atom may optionally bear on each said CH₂ or CH₃ group one or more substituents independently selected from hydroxy, amino, (1-6C)alkyl, (2-6C)alkenyl, (2-6C)alkynyl, (1-6C)alkoxy, (1-6C)alkylamino and di-[(1-6C)alkyl]amino;
or a pharmaceutically-acceptable salt thereof.

3. A compound of formula (I) according to claim 1 wherein R² is (1-6C)alkyl; or a pharmaceutically-acceptable salt thereof.

4. A compound of formula (I) according to claim 1 wherein R³ and R⁵ each represent hydrogen, halogeno or (1-6C)alkyl; or a pharmaceutically-acceptable salt thereof.

5. A compound of formula (I) according to claim 1 wherein
R⁴ is aryl or heteroaryl, which aryl or heteroaryl is substituted with (3-6C)cycloalkyl or heterocyclyl,
and which aryl or heteroaryl may further be optionally substituted by one or more substituents independently selected from halogeno, hydroxy, cyano, trifluoromethyl, trifluoromethoxy, amino, (1-6C)alkyl, (1-6C)alkoxy, (2-6C)alkenyl, (2-6C)alkynyl, (2-6C)alkanoyl, (1-6C)alkylthio, (1-6C)alkylsulphinyl, (1-6C)alkylsulphonyl, hydroxy-(2-6C)alkoxy, amino-(2-6C)alkoxy, cyano-(2-6C)alkoxy, (1-6C)alkylamino, di[(1-6C)alkyl]amino, (1-6C)alkylamino-(2-6C)alkoxy, di-[(1-6C)alkyl]amino-(2-6C)alkoxy, (1-6C)alkoxy-(2-6C)alkoxy, carbamoyl-(1-6C)alkoxy, N-(1-6C)alkylcarbamoyl-(1-6C)alkoxy, N-di-[(1-6C)alkyl]carbamoyl-(1-6C)alkoxy, amino-(1-6C)alkyl, (1-6C)alkylamino-(1-6C)alkyl, di[(1-6C)alkyl]amino-(1-6C)alkyl, hydroxy(1-6C)alkylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, hydroxy-(2-6C)alkylamino, cyano-(2-6C)alkylamino, halogeno-(2-6C)alkylamino, amino-(2-6C)alkylamino, (1-6C)alkoxy-(2-6C)alkylamino, (1-6C)alkylamino-(2-6C)alkylamino, di-[(1-6C)alkyl]amino-(2-6C)alkylamino, N-(1-6C)alkylsulphamoyl, N-di-[(1-6C)alkyl]sulphamoyl, (1-6C)alkanesulphonylamino, N-(1-6C)alkyl-(1-6C)alkanesulphonylamino, N-(1-6C)alkylcarbamoyl, N-di-[(1-6C)alkyl]carbamoyl, (1-6C)alkanoylamino, N-(1-6C)alkyl-(1-6C)alkanoylamino, carboxy, (1-6C)alkoxycarbonyl, (2-6C)alkanoyloxy, heteroaryl, heteroaryl-(1-6C)alkyl, heteroaryloxy, heteroarylthio, heteroarylsulphinyl, heteroarylsulphinoyl, heteroaryl-(1-6C)alkoxy, heteroarylamino, heterocyclyl, heterocyclyl-(1-6C)alkyl, heterocyclyloxy, heterocyclyl-(1-6C)alkoxy, heterocyclylamino, heterocyclylthio, aryl, aryl-(1-6C)alkyl, aryloxy, arylthio, arylthio, arylsulphinyl, aryl-(1-6C)alkoxy, arylamino, (3-6C)cycloalkyl, (3-6C)cycloalkyl-(1-6C)alkyl, (3-6C)cycloalkyloxy, (3-6C)cycloalkylthio, (3-6C)cycloalkyl-(1-6C)alkoxy and (3-6C)cycloalkylamino,
and wherein any heteroaryl, heterocyclyl, aryl or (3-6C)cycloalkyl group in a R⁴ substituent may be optionally substituted by one or more substituents independently selected from hydroxy, halogeno, (1-6C)alkyl, (2-6C)alkenyl, (2-6C)alkynyl, (3-6C)cycloalkyl, (3-6C)cycloalkyl-(1-6C)alkyl, (3-6C)cycloalkyl-(1-6C)alkoxy, (1-6C)alkoxy, (1-6C)alkylthio, carboxy, (1-6C)alkoxycarbonyl, (1-6C)alkoxycarbonyl-(1-6C)alkyl, N-(1-6C)alkylcarbamoyl, N,N-di-[(1-6C)alkyl]carbamoyl, (2-6C)alkanoyl, (2-6C)alkanoyloxy, amino, (1-6C)alkylamino, di-[(1-6C)alkyl]amino, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl and di-[(1-6C)alkyl]amino-(1-6C)alkyl,
and wherein any of the R⁴ substituents defined hereinbefore which comprises a CH₂ group which is attached to 2 carbon atoms or a CH₃ group which is attached to a carbon atom may optionally bear on each said CH₂ or CH₃ group one or more substituents independently selected from halogeno, hydroxy, amino, trifluoromethyl, trifluoromethoxy, oxo, carboxy, carbamoyl, acetamido, (1-6C)alkyl, (2-6C)alkenyl, (2-6C)alkynyl, (3-6C)cycloalkyl, (3-6C)cycloalkoxy, (1-6C)alkoxy, (1-6C)alkylamino, di-[(1-6C)alkyl]amino, hydroxy-(1-6C)alkyl, (1-6C)alkoxy-(1-6C)alkyl, halogeno-(1-6C)alkyl, (1-6C)alkoxy-(2-6C)alkoxy, (1-6C)alkoxycarbonyl, carbamoyl, N-(1-6C)alkylcarbamoyl, N,N-di-[(1-6C)alkyl]carbamoyl, (1-6C)alkylsulphonyl, N-(1-6C)alkylsulphamoyl, heteroaryl, heteroaryl-(1-6C)alkyl, heterocyclyl and heterocyclyloxy, and wherein any heterocyclyl group in a R⁴ substituent may optionally bear 1 or 2 oxo or thioxo substituent;
or a pharmaceutically-acceptable salt thereof.

6. A compound according to claim 1 of formula (IC), 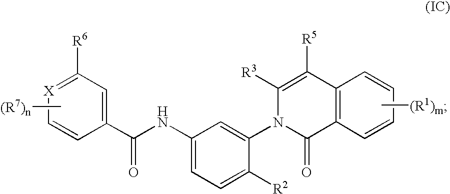
wherein
R¹, R² and m are as defined in claim 1,
X is CH or N,
R⁶ is halogeno, (3-6C)cycloalkyl, heteroaryl or heterocyclyl; which (3-6C)cycloalkyl, heteroaryl or heterocyclyl may be optionally substituted by one or more substituents independently selected from halogeno, hydroxy, (1-6C)alkyl, (1-6C)alkoxy, (1-6C)alkylsulphinyl, trifluoromethyl and trifluoromethoxy;
R⁷ is halogeno, hydroxy, cyano, trifluoromethyl, trifluoromethoxy, N,N-dialkylamino(1-6)alkylamino, N,N-dialkylamino(1-6)alkylthio, (1-6C)alkyl, (1-6C)alkoxy, (1-6C)alkylamino or di[(1-6C)alkyl]amino; and
n is 0, 1 or 2;
or a pharmaceutically-acceptable salt thereof.

7. A compound of formula (I) according to claim 1 selected from:
N-{4-Methyl-3-[1-oxo-7-(2-pyrrolidin-1-ylethoxy)isoquinolin-2(1H)-yl]phenyl}-3-piperidin-1-ylbenzamide,
N-{4-Methyl-3-[1-oxo-7-(2-pyrrolidin-1-ylethoxy)isoquinolin-2(1H)-yl]phenyl}-3-pyrrolidin-1-ylbenzamide.
3-Azepan-1-yl-N-{4-methyl-3-[1-oxo-7-(2-pyrrolidin-1-ylethoxy)isoquinolin-2(1H)-yl]phenyl}benzamide,
3-[(3R)-3-Hydroxypyrrolidin-1-yl]-N-{4-methyl-3-[1-oxo-7-(2-pyrrolidin-1-ylethoxy)isoquinolin-2(1H)-yl]phenyl}benzamide,
N-{4-Methyl-3-[1-oxo-7-(2-pyrrolidin-1-ylethoxy)isoquinolin-2(1H)-yl]phenyl}-2-pyrrolidin-1-ylisonicotinamide,
N-{4-Methyl-3-[1-oxo-7-(2-pyrrolidin-1-ylethoxy)isoquinolin-2(1H)-yl]phenyl}-3-(tetrahydro-2H-pyran-4-yl)benzamide,
N-{4-methyl-3-[1-oxo-7-(3-pyrrolidin-1-ylpropoxy)isoquinolin-2(1H)-yl]phenyl}-3-(2-thienyl)benzamide,
N-{3-[7-[3-(dimethylamino)propoxy]-1-oxoisoquinolin-2(1H)-yl]-4-methylphenyl}-3-(2-thienyl)benzamide,
N-{3-[7-[2-(dimethylamino)ethoxy]-1-oxoisoquinolin-2(1H)-yl]-4-methylphenyl}-2-(2-thienyl)isonicotinamide,
N-{4-Methyl-3-[1-oxo-7-(2-pyrrolidin-1-ylethoxy)isoquinolin-2(1H)-yl]phenyl}-2-(2-thienyl)isonicotinamide, and
N-{4-Methyl-3-[1-oxo-7-(3-pyrrolidin-1-ylpropoxy)isoquinolin-2(1H)-yl]phenyl}-3-(1,3-oxazol-5-yl)benzamide
or a pharmaceutically-acceptable salt thereof.

8. A process for preparing a compound of formula (I) according to claim 1, or pharmaceutically-acceptable salt thereof, which comprises
a) reacting a compound of formula (II) or a (1-6C)alkyl ester, acid anhydride or acid halide thereof, with a compound of formula (III) 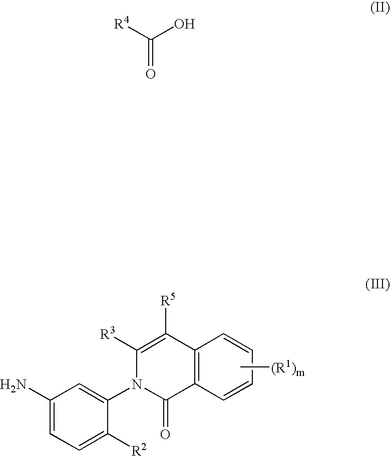
wherein R¹, m, R², R³, R⁴ and R⁵ are as defined in claim 1, or
b) dehydrating a compound of formula (XIII) wherein R¹, m, R², R³, R⁴ and R⁵ are as defined in claim 1 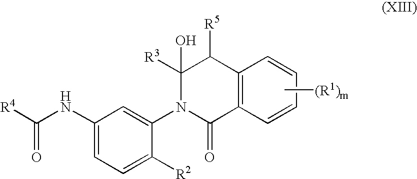
or
c) for compounds where m, R², R³, R⁴ and R⁵ are as defined in claim 1, and R¹ is amino-(2-6C)alkoxy, (1-6C)alkylamino-(2-6C)alkoxy, heterocyclyl-(2-6C)alkoxy or di-[(1-6C)alkyl]amino-(2,6C)alkoxy, reacting a compound of formula (XVIII), wherein L¹ represents a suitable leaving group and p is 1 to 5, 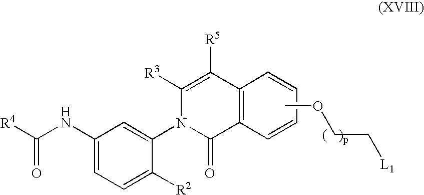
with an amine of formula HNWV wherein W and V are independently hydrogen, (1-6C)alkyl or, together with the nitrogen atom to which they are both attached, form a heterocyclyl ring that may optionally contain a further heteroatom,
and optionally after a), b) or c) carrying out one or more of the following:
(i) converting the compound to another compound of formula (I)
(ii) forming a pharmaceutically-acceptable salt of the compound.

9. A pharmaceutical composition which comprises a compound of formula (I) as claimed in any one of claims 1 to 7, or a pharmaceutically-acceptable salt thereof, in association with a pharmaceutically-acceptable diluent or carrier.

10. (canceled)

11. A method for the treatment of a disease condition selected from rheumatoid arthritis, asthma, chronic obstructive pulmonary disease, inflammatory bowel disease, multiple sclerosis, AIDS, septic shock, congestive heart failure, ischaemic heart disease or psoriasis, said method comprising administering to a warm-blooded animal in need thereof an effective amount of a compound of formula (I) as claimed in claim 1, or a pharmaceutically-acceptable salt thereof.

12. A pharmaceutical product comprising, in combination, a first active ingredient which is a compound of formula (I), or a pharmaceutically acceptable salt thereof, as claimed in claim 1, and at least one further active ingredient selected from:—
a phosphodiesterase inhibitor;
a β2 adrenoceptor agonist;
a modulator of chemokine receptor function;
a protease inhibitor;
a steroidal glucocorticoid receptor agonist;
an anticholinergic agent; and
a non-steroidal glucocorticoid receptor agonist.
